(19) United States
(12) Patent Application Publication
(10) Pub. No.: US 2018/0217163 A1
(43) Pub. Date: Aug. 2, 2018

(54) TREATMENT OF NEURODEGENERATIVE CONDITIONS USING PKC ACTIVATORS AFTER DETERMINING THE PRESENCE OF THE APOE4 ALLELE

(71) Applicant: Daniel L. ALKON, Chevy Chase, MD (US)

(72) Inventors: Daniel L. Alkon, Chevy Chase, MD (US); Abhik Sen, Morgantown, WV (US); Thomas Nelson, Morgantown, WV (US)

(21) Appl. No.: 15/572,946

(22) PCT Filed: May 11, 2016
(86) PCT No.: PCT/US2016/031942

§ 371 Date: Nov. 9, 2017

Related U.S. Application Data
(60) Provisional application No. 62/159,691, filed on May 11, 2015.

(51) Int. Cl. G01N 33/68; A61K 31/365; C12Q 1/6883; A61P 25/28

(52) U.S. Cl. CPC G01N 33/6896; A61K 31/365; C12Q 1/6883; A61P 25/28; G01N 2800/52; C12Q 2600/156; G01N 2333/912; G01N 2333/98; G01N 2800/50; C12Q 2600/106

(57) ABSTRACT

The present disclosure provides for methods of treating a neurodegenerative condition, as well as methods for assessing the risk of developing a neurodegenerative condition, and assessing treatment efficacy in subjects who are carriers of the ApoE4 allele. Also disclosed is a method for diagnosing a neurodegenerative disorder.

This application claims priority to U.S. Provisional Application 62/159,691, filed May 11, 2015, the entire contents of which are incorporated herein by reference.

The apolipoprotein E4 allele (ApoE4) is a major risk factor for sporadic and late-onset Alzheimer's disease (LOAD), as well as other neurodegenerative conditions. In Alzheimer's disease (AD) and healthy aged controls, ApoE4 levels are inversely correlated to dendritic spine density in the hippocampus. In fact, the risk of AD is 2- to 3-fold higher in patients with one ApoE4 allele and 12-fold higher in patients with two ApoE4 alleles (Michaelson D. M., APOE epsilon4: the most prevalent yet understudied risk factor for Alzheimer's disease, Alzheimers Dement, 10:861-868, 2014). Both patient types, for example, one allele or two allele carriers such as homozygous and/or heterozygous, are carriers for the ApoE4 allele.

ApoE, which in the brain is produced mainly in astrocytes, is a cholesterol-transporting protein and a major determinant of synapse formation and remodeling (Pfrieger, F. W., Cholesterol homeostasis and function in neurons of the central nervous system, Cell Mol Life Sci, 60:1158-1171 2003; Bu, G., Apolipoprotein E and its receptors in Alzheimer's disease: pathways, pathogenesis and therapy. Nat Rev Neurosci., 10:333-344 2009). ApoE is also a ligand for lipoprotein receptors and thus may have a role in promoting amyloid-β (Aβ) clearance through the blood-brain barrier or the blood-CSF barrier. There are many functional differences between ApoE3 and ApoE4. For instance, ApoE4 increases AP deposition in brain (Verghese et al., Apolipoprotein E in Alzheimer's disease and other neurological disorders, Lancet Neurol., 10: 241-252, 2011; Liu et al., Apolipoprotein E and Alzheimer disease: risk, mechanisms and therapy, Nat Rev Neurol., 9:106-118, 2013) and knock-in transgenic mice containing human ApoE4 allele showed reduced synaptic transmission compared to mice with the human ApoE3 allele (Klein et al., Progressive loss of synaptic integrity in human apolipoprotein E4 targeted replacement mice and attenuation by apolipoprotein E2, Neuroscience, 171:1265-1272 2010). Transcriptome-wide differential gene expression analysis further showed that ApoE4 produces changes in gene expression similar to those found in patients with LOAD. (Rhinn et al., Integrative genomics identifies APOE epsilon4 effectors in Alzheimer's disease, Nature, 500:45-50, 2013). Previous studies by the present inventors show that ApoE3 acts via protein kinase C ε (PKCε) to protect primary neurons against AP-induced cell death and induces synaptogenesis, whereas ApoE4 does not (Sen et al., Apolipoprotein E3 (ApoE3) but not ApoE4 protects against synaptic loss through increased expression of protein kinase C epsilon, J. Biol. Chem., 287:15947-15958, 2012).

Brain derived neurotrophic factor (BDNF) is a relevant factor in synaptic repair and plasticity. Although evidence for BDNF polymorphisms in AD is still inconclusive, synaptic loss is the single most important correlate of AD. Lower BDNF levels are associated with ApoE4 in AD cases with apathy, a noncognitive symptom common to many forms of dementia (Alvarez et al., Apathy and APOE4 are associated with reduced BDNF levels in Alzheimer's disease, J. Alzheimers Dis., 42:1347-1355, 2014). BDNF expression is regulated by at least nine promoters (Aid et al., Mouse and rat BDNF gene structure and expression revisited, J. Neurosci. Res.; 85:525-535, 2007; Pruunsild et al., Dissecting the human BDNF locus: bidirectional transcription, complex splicing, and multiple promoters, Genomics, 90:397-406, 2007), of which promoter IV (PIV) is most responsive to neuronal activity (Tao et al., Ca2 influx regulates BDNF transcription by a CREB family transcription factor-dependent mechanism, Neuron, 20:709-726, 1998). PKCε, which is decreased in AD (Hongpaisan et al., PKC epsilon activation prevents synaptic loss, Abeta elevation, and cognitive deficits in Alzheimer's disease transgenic mice, J. Neurosci., 31:630-643, 2011; Khan et al., PKCepsilon deficits in Alzheimer's disease brains and skin fibroblasts, J. Alzheimers Dis., 43:491-509, 2015), also regulates BDNF expression (Lim and Alkon, 2012; Corbett et al., 2013; Hongpaisan et al., PKC activation during training restores mushroom spine synapses and memory in the aged rat, Neurobiol. Dis., 55:44-62, 2013; Neumann et al., Increased BDNF protein expression after ischemic or PKC epsilon preconditioning promotes electrophysiologic changes that lead to neuroprotection, J. Cereb. Blood Flow Metab., 35:121-130, 2015). BDNF expression is also regulated in part by exon-specific epigenetic modifications.

Recent studies show that histone acetylation and de-acetylation are abnormal in several neurodegenerative conditions, including AD (Saha et al., HATs and HDACs in neurodegeneration: a tale of disconcerted acetylation homeostasis, Cell Death Differ., 13:539-550, 2006; Kramer et al., Genetic and epigenetic defects in mental retardation, Int. J. Biochem. Cell Biol., 41:96-107, 2009; Mai et al., Histone deacetylase inhibitors and neurodegenerative disorders: holding the promise, Curr. Pharm. Des., 15:3940-3957, 2009; Fischer et al., Targeting the correct HDAC(s) to treat cognitive disorders, Trends Pharmacol. Sci., 31:605-617, 2010; Gräff et al., An epigenetic blockade of cognitive functions in the neurodegenerating brain, Nature, 483:222-226, 2012). Postmortem studies reported that histone deacetylase 2 (HDAC2) is increased in the hippocampus of AD patients (Gräff et al., 2012). Class II HDAC6 levels are also elevated in AD cortex and hippocampus (Ding et al., Histone deacetylase 6 interacts with the microtubule-associated protein tau, J. Neurochem., 106:2119-2130, 2008). Nuclear staining showed that HDAC4 levels in CA1 neurons increases with increase in AD severity (Herrup et al., The role of ATM and DNA damage in neurons: upstream and downstream connections, DNA Repair (Amst), 12:600-604, 2013). Accordingly, HDAC inhibitors are reported to improve memory and cognition (Fischer et al., Recovery of learning and memory is associated with chromatin remodeling, Nature, 447:178-182, 2007; Kilgore et al., Inhibitors of class 1 histone deacetylases reverse contextual memory deficits in a mouse model of Alzheimer's disease, Neuropsychopharmacology, 35:870-880, 2010) by inducing histone H3 and H4 acetylation of BDNF promoters (Bredy et al., Histone modifications around individual BDNF gene promoters in prefrontal cortex are associated with extinction of conditioned fear, Learn Mem., 14:268-276, 2007; Ishimaru et al., Differential epigenetic regulation of BDNF and NT-3 genes by trichostatin A and 5-aza-2-deoxycytidine in Neuro-2a cells, Biochem. Biophys. Res. Commun., 394:173-177, 2010; Boulle et al., Epigenetic regulation of the BDNF gene: implications for psychiatric disorders, Mol. Psychiatry, 17:584-596, 2012). Additionally, Class II HDAC inhibitors also induce BDNF PIV activity (Koppel and Timmusk, Differential regulation of BDNF expression in cortical neurons by class-selective histone deacetylase inhibitors, Neuropharmacology, 75:106-115, 2013).

The present inventors investigated herein the role of ApoE isoforms and PKC on nuclear translocation of HDACs and BDNF expression in neuronal cells in the presence or absence of amyloid-β amylospheroids (ASPDs) in order to mimic AD in vitro. ApoE3 and ApoE4 differentially regulate gene transcription in AD by modulating histone acetylation through HDACs in the brain. As a result, the present disclosure supports treatment with one or more PKC activators, such as macrocyclic lactones, in a patient deficient in PKCε production and/or processing such as in patients homo- or heterozygous for ApoE4. Treatment results increased PKCε production, mRNA protein levels, and membrane association. Those thereby elevated BDNF and other synaptic growth factors, increased synaptogenesis, and enhanced cognitive functions.

The present invention relates to a method for treating a neurodegenerative disorder as well as to methods for assessing treatment efficacy of a neurodegenerative disease, diagnosing a neurodegenerative disorder, and a method for assessing a risk of developing a neurodegenerative condition and the use of PKC activators as therapeutics for the treatment of a neurodegenerative disorder, such as Alzheimer's disease.

In one embodiment, the method treating a neurodegenerative disorder in a subject comprises obtaining a biological sample from the subject, identifying whether the subject is a carrier of the ApoE4 allele and administering to the subject, if the subject is a carrier of the ApoE4 allele, a therapeutically effective amount of a PKC activator.

Neurodegenerative disorders treated by the method are Alzheimer's disease, chronic traumatic encephalopathy (CTE), Parkinson's disease, multiple sclerosis, and traumatic brain injury. In one embodiment, treatment is effected to a person with Alzheimer's disease, for example, a persons with sporadic Alzheimer's disease or late-onset Alzheimer's disease.

Biological samples for use with the inventive method can be chosen from skin cells, fibroblasts, blood cells, olfactory neurons, and buccal mucosal cells.

Treatment by the method of the present disclosure is effected by administering a therapeutically effective amount of a PKC activator to a subject that is a carrier of the ApoE4 allele. According to one embodiment, the PKC activator is a compound chosen from macrocyclic lactones, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

According to an aspect of this embodiment, the PKC activator is the macrocyclic lactone bryostatin. In one embodiment, the wherein the bryostatin is chosen from bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

The PKC activator can be administered to a subject that is a homozygous carrier of the Apolipoprotein E ε4 allele or a subject that is a heterozygous carrier of the Apolipoprotein E ε4 allele every week for a period of time ranging from about two weeks to about 4 weeks. A therapeutically effective dose of the PKC activator is about 5-20 μg/sq·m/week.

According to another embodiment, the present disclosure provides for a method for assessing treatment efficacy of a neurodegenerative disease in a subject by administering to the subject with a neurodegenerative disease one or more therapeutically effective active agents, then obtaining a first biological sample and a second biological sample from the subject at different time points during the treatment, followed by measuring the level of PKC-ε in the first and second samples; and then comparing the levels of PKC-ε in the first and second samples, wherein a higher level of PKC-ε in the second sample compared to the first sample is an indicator of efficacy of the treatment.

In an embodiment of this method, wherein the first biological sample is obtained before administering treatment, and the second biological sample is obtained after administering treatment.

According to a further embodiment of this method, treatment is administered for a period of time from 2 weeks, 3 weeks, 4 weeks, 5 weeks, and 6 weeks.

In one embodiment, the active agent is a PKC activator. Illustrative PKC activators suitable for use with the disclosed method include macrocyclic lactones, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

In one embodiment, the PKC activator is the macrocyclic lactone bryostatin.

In yet another embodiment, the disclosure provides a method for diagnosing a neurodegenerative disorder in a subject by obtaining a biological sample form the subject, then lysing the biological sample to obtain a lysate and differentially fractionating the lysate to obtain a cytoplasmic fraction and a nuclear fraction prior to measuring the ratio of HDAC4 or HDAC6 to total HDAC in the nuclear fraction. According to this method, the subject has neurodegenerative disorder if the ratio of HDAC4 to total nuclear HDAC or the ratio of HDAC6 to total nuclear HDAC is in the range from 0.5 to 0.95.

In yet another embodiment, the disclosure provides a method for assessing a risk of developing a neurodegenerative condition in a subject by obtaining a biological sample from the subject, then lysing the biological sample to obtain a lysate and differentially fractionating the lysate to obtain a cytoplasmic fraction and a nuclear fraction prior to measuring the level of a HDAC4 or a HDAC6 in the cytoplasmic fraction and the nuclear fraction. According to this method, the risk of developing the neurodegenerative condition is greater if the level of HDAC4 or HDAC6 in the nuclear fraction is greater than their corresponding levels in the cytoplasmic fraction.

In one embodiment, the level of HDAC4 or HDAC6 in the nuclear fraction of a subject at risk for developing a neurodegenerative condition is 1.5-fold to 2.5 fold greater than the level of HDAC4 or HDAC6 in the cytoplasmic fraction.

### BRIEF DESCRIPTION OF THE FIGURES

FIGS. 1A and 1B: Comparison of ApoE3, ApoE4 and histone 3 acetylation in SH-SY5Y cells.

FIGS. 2A-C: Comparison of ApoE3, ApoE4, HDAC4 and HDAC6 translocation in SH-SY5Y cells.

FIGS. 3A-D: Comparison of ApoE3, ApoE4, HDAC4 and HDAC6 translocation in primary human neurons.

FIGS. 4A and 4B: Comparison of ApoE3, ApoE4, HDAC4 and HDAC6 nuclear localization in the hippocampus of transgenic mice.

FIGS. 5A-F: Comparison of ApoE3, ApoE4, HDAC4 and HDAC6 nuclear translocation in SH-SY5Y cells pre-treated with receptor binding protein RAP.

FIGS. 6A-I: PKCε, PKCα, and PKCδ mRNA levels in SH-SY5Y cells treated with cholesterol with or without ApoE3 or ApoE4.

FIGS. 7A-G: BDNF expression in SH-SY5Y cells by ApoE3 and ApoE4.

FIGS. 8A-H: SH-SY5Y cells treated with cholesterol and ApoE3 or cholesterol and ApoE4 in the presence or absence of ASPDs.

FIG. 9: ApoE-isoform-mediated regulation of gene expression.

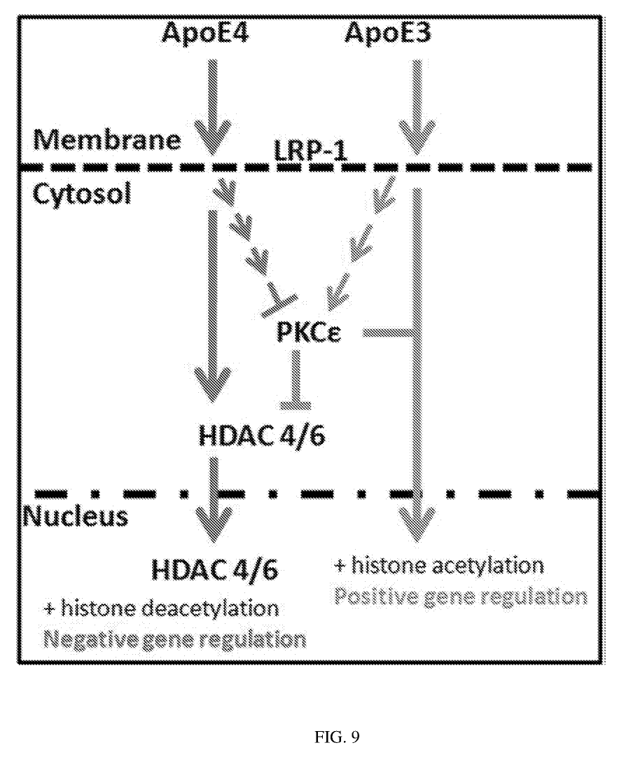

FIG. 10: BR-122 activates PKC in primary neurons.

FIG. 11: Bryostatin activates PKCε in brain of mice.

FIG. 12: Phase IIa Clinical Trial shows Bryostatin to increase synthesis of PKCε.

FIG. 13: Blood levels of PKCε at 1 h., following administration of bryostatin.

FIG. 14: Phase IIa Clinical Trial shows increased levels of PKCε at 1 h after onset of infusion of bryostatin. In red, the figure illustrates increasing slope for the line for PKCε up to 1 h peak.

FIGS. 15A and 15B: PKC was constitutively more activated in mice expressing hApoE3, as indicated by an increased percentage of total PKC in the particulate fraction (28.6±1.1%, mean±SE), compared with transgenic mice expressing human ApoE4 (21.6±1.0%) or wild-type mice (23.5±0.5%).

FIG. 16: Bryostatin infusion improves cognition by increasing the mini-mental state examination score (MMSE).

### DESCRIPTION

As used herein, the singular forms “a,” “an,” and “the” include plural reference.

As used herein, “protein kinase C activator” or “PKC activator” refers to a substance that increases the rate of the reaction catalyzed by PKC. PKC activators can be non-specific or specific activators. A specific activator activates one PKC isoform, e.g., PKC-ε (epsilon), to a greater detectable extent than another PKC isoform.

As used herein, the term “fatty acid” refers to a compound composed of a hydrocarbon chain and ending in a free acid, an acid salt, or an ester. When not specified, the term “fatty acid” is meant to encompass all three forms. Those skilled in the art understand that certain expressions are interchangeable. For example, “methyl ester of linolenic acid” is the same as “linolenic acid methyl ester,” which is the same as “linolenic acid in the methyl ester form.”

As used herein, the term “cyclopropanated” or “CP” refers to a compound wherein at least one carbon-carbon double bond in the molecule has been replaced with a cyclopropane group. The cyclopropyl group may be in cis or trans configuration. Unless otherwise indicated, it should be understood that the cyclopropyl group is in the cis configuration. Compounds with multiple carbon-carbon double bonds have many cyclopropanated forms. For example, a polyunsaturated compound in which only one double bond has been cyclopropanated would be said to be in “CP1 form.” Similarly, “CP6 form” indicates that six double bonds are cyclopropanated.

For example, docosahexaenoic acid (“DHA”) methyl ester has six carbon-carbon double bonds and thus can have one to six cyclopropane rings. Shown below are the CP1 and CP6 forms. With respect to compounds that are not completely cyclopropanated (e.g. DHA-CP1), the cyclopropane group(s) can occur at any of the carbon-carbon double bonds.

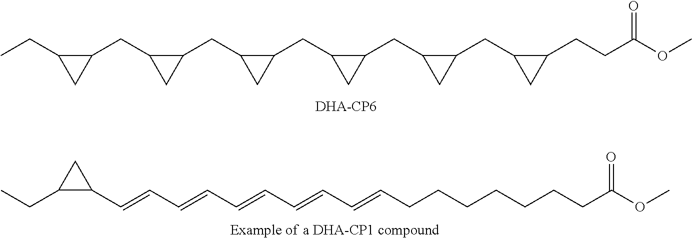

As used herein, the word “cholesterol” refers to cholesterol and derivatives thereof. For example, “cholesterol” is understood to include the dihydrocholesterol species.

As used herein, the word “synaptogenesis” refers to a process involving the formation of synapses.

As used herein, the word “synaptic networks” refer to a multiplicity of neurons and synaptic connections between the individual neurons. Synaptic networks may include extensive branching with multiple interactions. Synaptic networks can be recognized, for example, by confocal visualization, electron microscopic visualization, and electrophysiologic recordings.

The phrase “pharmaceutically acceptable” refers to molecular entities and compositions that are physiologically tolerable and do not typically produce untoward reactions when administered to a subject. For example, as used herein, the term “pharmaceutically acceptable” means approved by a regulatory agency of the federal or a state government or listed in the U.S. Pharmacopeia or other generally recognized pharmacopeia for use in animals, and more particularly in humans. The term “pharmaceutically acceptable carrier” means a chemical composition with which the active ingredient may be combined and which, following the combination, can be used to administer the active ingredient to a subject and can refer to a diluent, adjuvant, excipient, or vehicle with which the compound is administered.

The terms “therapeutically effective dose” and “effective amount” refer to an amount of a therapeutic agent that results in a measurable therapeutic response. A therapeutic response may be any response that a user (e.g., a clinician) will recognize as an effective response to the therapy, including improvement of symptoms and surrogate clinical markers. Thus, a therapeutic response will generally be an amelioration or inhibition of one or more symptoms of a disease or condition. A measurable therapeutic response also includes a finding that a symptom or disease is prevented or has a delayed onset, or is otherwise attenuated by the therapeutic agent.

The terms “approximately” and “about” mean to be nearly the same as a referenced number or value including an acceptable degree of error for the quantity measured given the nature or precision of the measurements. As used herein, the terms “approximately” and “about” should be generally understood to encompass ±20% of a specified amount, frequency or value. Numerical quantities given herein are approximate unless stated otherwise, meaning that term “about” or “approximately” can be inferred when not expressly stated.

The terms “administer,” “administration,” or “administering” as used herein refer to (1) providing, giving, dosing and/or prescribing by either a health practitioner or his authorized agent or under his direction a composition according to the disclosure, and (2) putting into, taking or consuming by the patient or person himself or herself, a composition according to the disclosure. As used herein, “administration” of a composition includes any route of administration, including oral, intravenous, subcutaneous, intraperitoneal, and intramuscular.

The present disclosure relates to methods for treating and/or reducing the risk of developing a neurodegenerative disorder, such as Alzheimer's disease (e.g., sporadic or late-onset), chronic traumatic encephalopathy (CTE), Parkinson's disease, multiple sclerosis, and traumatic brain injury. Because direct access to brains of living humans is impossible, assessing the risk of developing a neurodegenerative condition in a subject is difficult.

Even more challenging is early diagnoses of the onset of a neurodegenerative condition. The present invention provides a method for assessing the risk of developing a neurodegenerative condition as well as a method for diagnosing a neurodegenerative disorder in a subject. The disclosed methods are based on the discovery that patients with one or more copies of the ApoE4 allele are at an increased risk for developing AD, particularly late-onset Alzheimer's disease (LOAD).

Also described is a method for treating a subject diagnosed with a neurodegenerative disorder and a method of assessing treatment efficacy in subjects with a neurodegenerative disease.

Apolipoprotein E (ApoE) is known to promote amyloid-β (Aβ) clearance through the blood-brain barrier or the blood-CSF barrier. While the ApoE3 isoform protects primary neurons against Aβ-induced cell death and promotes synaptogenesis, ApoE4 isoform levels are known to correlate with Aβ deposition in brain and an increased risk of developing Alzheimer's disease (AD). In fact, the risk for developing AD is 2- to 3-fold greater in patients with one ApoE4 allele and about 12-fold greater in patients with two ApoE4 alleles.

In one aspect, the disclosure provides a method for treating a neurodegenerative disorder in a subject by administering to the subject identified to be a carrier of the ApoE4 allele, a therapeutically effective amount of a PKC activator.

Neurodegenerative disorders treated by the disclosed method include Alzheimer's disease, chronic traumatic encephalopathy (CTE), Parkinson's disease, multiple sclerosis, and traumatic brain injury. In a preferred aspect, the neurodegenerative disorder is Alzheimer's disease, for example, sporadic Alzheimer's disease or late-onset Alzheimer's disease.

The disclosed method is suitable for treating subject who is a heterozygous carrier of the ApoE4 allele, or a subject who is a homozygous carrier of the ApoE4 allele. Subjects who are homozygous carriers of the allele are at a greater risk of disease progression.

The disclosure also provides methods for assessing treatment efficacy by comparing the levels of PKC-ε in a first and a second biological sample obtained from the subject at two different time points during treatment. In one aspect of this method, a higher level of PKC-ε in the second sample compared to the first sample is an indicator of efficacy of the treatment.

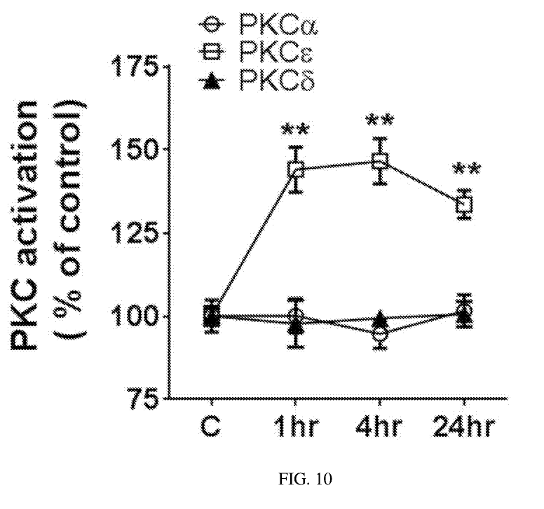

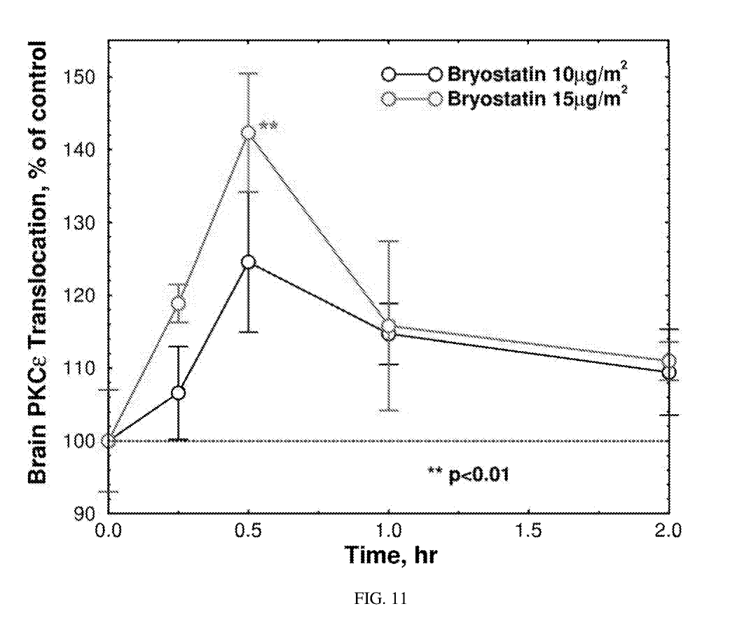

Treatment using a PKC activator according to this method can be for a week or over multiple weeks or months. In one embodiment the PKC activator is bryostatin. As shown in FIGS. 10 and 11, administration of BR-122, an analog of bryostatin, increases PKC-ε levels in primary neurons. Similarly the administration of bryostatin to mice increased the PKC-ε levels in brain of mice.

In another embodiment, the disclosure provides a method for diagnosing a neurodegenerative disorder in a subject based on the nuclear ratio of HDAC4 or HDAC6 to total HDAC in the nucleus of a cell from a biological sample of the subject.

In one embodiment, a diagnosis of a neurodegenerative disorder is confirmed when the ratio of HDAC4 to total nuclear HDAC or the ratio of HDAC6 to total nuclear HDAC is in the range from 0.5 to 0.95. In one embodiment, the ratio of HDAC4 to total nuclear HDAC or the ratio of HDAC6 to total nuclear HDAC is in the range from 0.6 to 0.95, 0.7 to 0.95, or 0.8 to 0.95.

According to an aspect of this embodiment, the ratio of HDAC4 to total nuclear HDAC or the ratio of HDAC6 to total nuclear HDAC is 0.55, 0.6, 0.65, 0.7, 0.75, 0.8, 0.85, or 0.9.

In yet another aspect, the disclosed methods for assessing a risk of a neurodegenerative disorder comprise obtaining a biological sample from a patient at risk of developing such a condition, and measuring the level of a HDAC4 or a HDAC6 in the cytoplasmic fraction and the nuclear fraction of a lysate of the biological sample.

In one embodiment, the risk of developing a neurodegenerative disorder is high when the level of HDAC4 or HDAC6 in the nuclear fraction is about 1.5-fold, 1.75-fold, 1.80-fold, 1.85-fold, 1.9-fold, 1.95-fold, 2-fold, 2.1-fold, 2.2-fold, 2.3-fold, 2.4-fold, or 2.5-fold greater than the level of HDAC4 or HDAC6 in the cytoplasmic fraction.

In one embodiment, the level of HDAC4 or HDAC6 in the nuclear fraction is 1.5-fold greater than the level of HDAC4 or HDAC6 in the cytoplasmic fraction.

In another embodiment, the level of HDAC4 or HDAC6 in the nuclear fraction is 2.0-fold greater than the level of HDAC4 or HDAC6 in the cytoplasmic fraction.

In yet another embodiment, the level of HDAC4 or HDAC6 in the nuclear fraction is 2.5-fold greater than the level of HDAC4 or HDAC6 in the cytoplasmic fraction.

The biological sample can be any viable cell, that is, a cell obtained from a living donor, a sample tissue or cultured cells. For example, biological tissue is obtained and cells are separated from the tissue by methods known in the relevant art. Exemplary biological samples include without limitation skin sample cells, fibroblasts, blood cells, olfactory neurons, buccal mucosal cells, or any peripheral tissue cells obtained by non-invasive methods. The biological sample, however, can be a tissue or cells obtained from a patient using a minimally invasive procedure such as a spinal tap or lumbar puncture.

In an embodiment, the cells are blood cells obtained by drawing blood from the peripheral vein of a subject. Illustrative of the category “blood cells” are erythrocytes, lymphocytes, including B lymphocytes, T lymphocytes, and platelets.

According to another embodiment, punch skin biopsy is used to obtain skin fibroblasts from a subject. The cell density in the biological sample is readily determined using a Coulter counter and cell viability is determined, if necessary, by the Trypan blue dye exclusion method.

### Role of ApoE3 and ApoE4 in Reducing Risk of a Neurodegenerative Disease

ApoE4 is a biomarker for assessing the risk of developing a neurodegenerative condition, such as AD. For example, the present disclosure relates to the observation that the risk of developing a neurodegenerative condition is about 10-fold greater in patients carrying two copies of the ApoE4 allele compared to a patient with one copy of the ApoE4 allele.

Thus, methods are disclosed for treating and/or reducing the risk of a neurodegenerative condition. Although the exact mechanism for the onset and progression of AD is not well understood, deacetylation of histone H3 in neurons is implicated to play a role in disease pathogenesis.

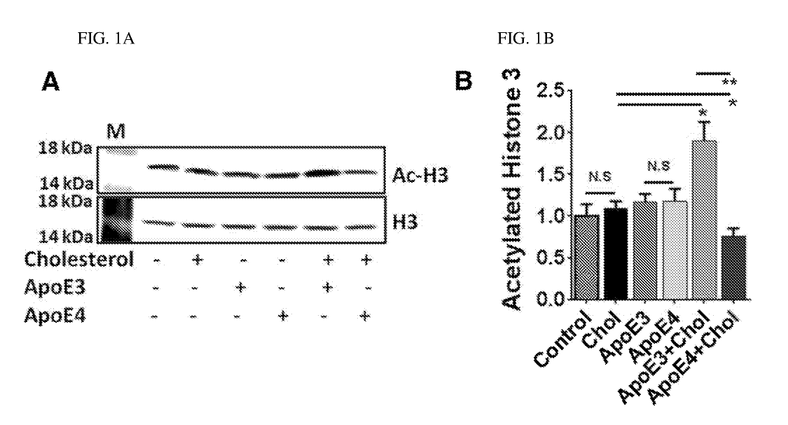

Surprisingly, ApoE3 and ApoE4 differentially regulate the acetylation state of histones, for example histone H3, in neurons. FIGS. 1A and 1B, illustrate the level of acetylation of lysine 9/14 (H3K9/14ac) in histone 3 for SH-SY5Y neuroblastoma cells treated with cholesterol, ApoE3, ApoE4, ApoE3 +cholesterol (ApoE3+Chol), or ApoE4+cholesterol (ApoE4+Chol).

As illustrated in FIG. 1, treatment of SH-SY5Y neuroblastoma cells with ApoE3+Chol for 24 h, increased H3K9/14ac by 89%, whereas levels of H3K9/14ac decreased by 25% in cells treated with ApoE4+Chol compared to control cholesterol-treated SH-SY5Y cells (F_{(5,12)}=7.33; ANOVA, p<0.0023). In fact, acetylated H3K9/14 was 2.4-fold higher in ApoE3+Chol treated cells than in ApoE4+Chol-treated cells (t test, p<0.005; FIG. 1B). Treatment of cultured SH-SY5Y neuroblastoma cells with cholesterol, ApoE3, or ApoE4 alone had no effect on acetylation of H3K9/14.

To further investigate whether the acetylation state of histones depends at least in part on their localization, that is, whether a histone is present within the cytoplasm or nucleus of SH-SY5Y cells, cytosolic and nuclear fractions were prepared using SH-SY5Y cells pre-treated with cholesterol, ApoE3, ApoE4, ApoE3+Chol, or ApoE4+Chol for 24 h. Immunoblot analysis of Class I HDAC's, namely, HDAC1, HDAC2, and HDAC3, and Class II HDAC's, namely, HDAC4, HDACS, and HDAC6 expressed in the brain, showed that class I HDACs, are primarily localized in the nucleus. The percent nuclear localization for HDAC1 is 90%, while the percent nuclear localization for HDAC2 and HDAC3 are 80% and 50% respectively. Moreover, Class I HDAC' s showed no significant change in localization behavior in response to treatment with ApoE3+Chol or ApoE4+Chol (data not shown).

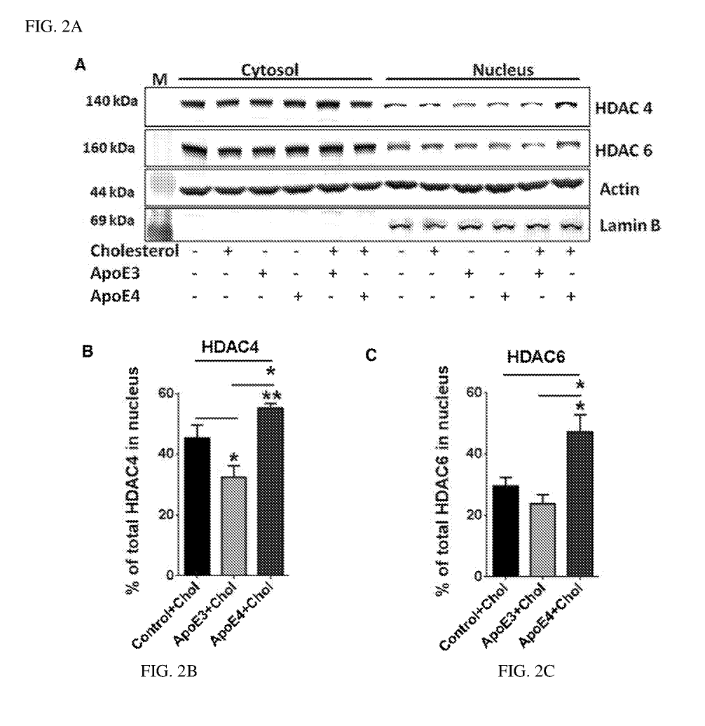

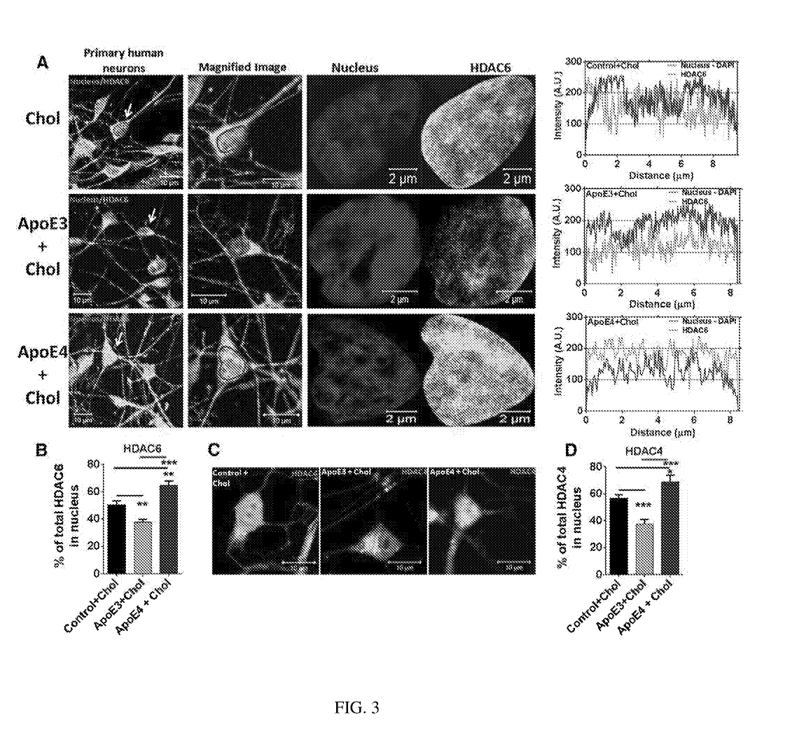

In contrast, increased nuclear translocation was observed for Class II HDACs (see, FIG. 2A). For example, a significant increase in nuclear translocation was observed for HDAC4 (F_{(2,8)}=11.01; ANOVA, p<0.01) in ApoE4+Chol treated cells (55.3±1.4%) compared with ApoE3+Chol (32.4±3.8%; p<0.005) and cholesterol (45.3±4.4%; P<0.05)-treated cells (see FIG. 2B). While HDAC5 showed no significant change in localization with treatment (data not shown), ApoE4+Chol caused a 2-fold increasein nuclear translocation of HDAC6 (47.2±5.6%, F_{(2,8)}=9.2; ANOVA, p<0.01) compared to cells treated with ApoE3+Chol (23.8±3.1%; p<0.01) and cholesterol alone (29.6±2.8%; FIG. 2C). No measurable change in nuclear translocation of HDAC4 or HDAC6 was observed in cells treated with cholesterol, ApoE3, or ApoE4 alone (see FIG. 2A). Further proof for ApoE4+Chol-induced HDAC6 and HDAC4 nuclear translocation in primary human neurons was obtained by confocal microscopy. Primary human neurons were treated with either cholesterol, ApoE3+Chol or ApoE4+Chol for 24 h. Confocal microscopy of ApoE4+Chol treated cells showed a 32% increase in nuclear fluorescence for HDAC6 (155.8±18.28, n=30 cells). In contrast, ApoE3+Chol treated cells showed reduced nuclear fluorescence for HDAC6. The percent decrease in nuclear fluorescent intensity for ApoE3+Chol treated cells was 41% (69.22±5.87, n=31 cells) compared to cholesterol treated cells used as control (117.4±10.39, n=28; FIG. 3A).

The results from confocal microscopy indicate that overall, a lower amount of HDAC6 is localized in the nucleus of ApoE3+Chol-treated neurons compared to ApoE4+Chol (37.7±2.14%; p<0.005, n=8 experiments, 40 cells vs 64.7±3.39%; p<0.001, n=8 experiments, 40 cells) treated neurons or compared to cholesterol treated neuronal cells (50.4±3.0%; F_{(2,21)}=21.6; ANOVA, p<0.0001, n=8 experiments, 40 cells; FIG. 3B). Similar results were observed for HDAC4, where confocal microscopy showed lower amounts of HDAC4 localization in neurons treated with ApoE3+Chol compared to neurons treated with cholesterol alone or ApoE4+Chol (Cholesterol alone=57.7±2.8%; ApoE4+Chol=69.2±4.8%; ApoE3+Chol=37.8±3.1%, F_{(2,21)}=18.2; ANOVA, p<0.0001, n=8 experiments, 40 cells; see FIG. 3C,D).

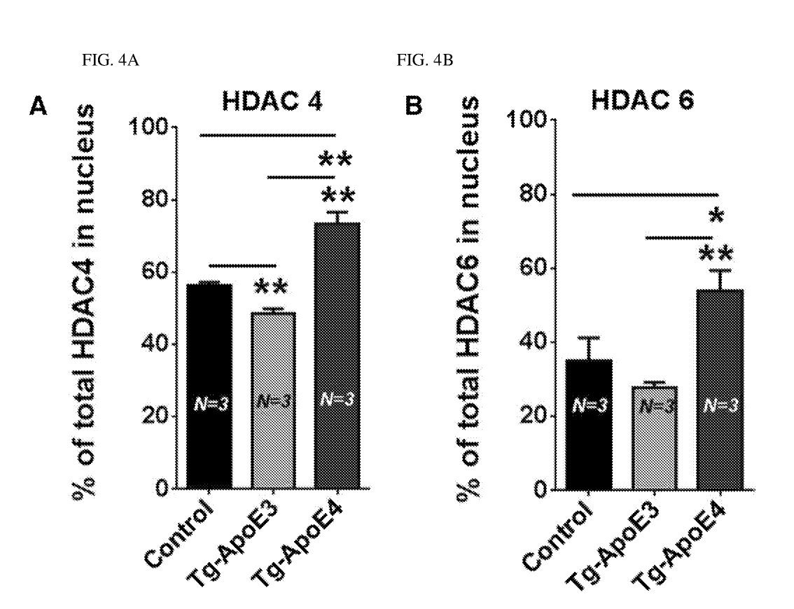

Further support for the role of ApoE4 in promoting the translocation of Class II HDAC's into the nucleus comes from studies that involved transgenic C57BL/6 mice. Two groups of mice were used for the study. The first group of mice were carriers of a human allele for ApoE3 and the second group of mice were carriers of a human allele for ApoE4. As illustrated in FIG. 4A, the total amount of nuclear HDAC4 in the hippocampus of ApoE4 transgenic mice, was higher (73.3±3.3% nuclear, n=3) than the total amount of nuclear HDAC4 in the hippocampus of ApoE3 transgenic mice (48.5±1.4%, n=3; t test, p<0.003) or control mice (56.6±0.7%, n=3; t test, p<0.004; FIG. 4A). Similarly, the total amount of nuclear HDAC6 was higher in ApoE4 transgenic mice (54.5±5.4%) compared to ApoE3 transgenic mice (27.9±1.3%; t test, p<0.005) and control mice (34.9±6.3%; t test, p<0.05; FIG. 4B). From these results, ApoE3 and ApoE4 exert differential effects on nucleo-cytoplasmic shuttling of HDAC4 and HDAC6. In certain aspects of the inventive method, the risk of a neurodegenerative disorder is greater if the levels of HDAC4 or HDAC6 in the nucleus of a cell of the biological sample is greater than 50% of the total nuclear HDAC's, greater than 55% of the total nuclear HDAC's, greater than 60% of the total nuclear HDAC's, greater than 65% of the total nuclear HDAC's, greater than 70% of the total nuclear HDAC's, greater than 75% of the total nuclear HDAC's, greater than 80% of the total nuclear HDAC's, greater than 85% of the total nuclear HDAC's, greater than 90% of the total nuclear HDAC's, or greater than 95% of the total nuclear HDAC's.

According to this aspect, the level of HDAC4 or HDAC5 in the nucleus is determined by an immunoassay, for example a radioimmunoassay, a Western blot assay, an immunofluorescence assay, an enzyme immunoassay, an immunoprecipitation assay, an immunohistochemical assay, an immunoelectrophoretic assay, chemiluminescence assay, dot-blot assay or a slot blot assay.

In certain aspects, the risk of a neurodegenerative disorder is lowered by treating a patient with a PKC activator. In certain embodiments, PKC activators are macrocyclic lactones, e.g., the bryostatin and neristatin classes, which act to stimulate PKC. Macrocyclic lactones (also known as macrolides) generally comprise 14-, 15-, or 16-membered lactone rings. Macrolides belong to the polyketide class of natural products. Macrocyclic lactones and derivatives thereof are described, for example, in U.S. Pat. Nos. 6,187,568; 6,043,270; 5,393,897; 5,072,004; 5,196,447; 4,833,257; and 4,611,066; and 4,560,774; each incorporated by reference herein in its entirety. Those patents describe various compounds and various uses for macrocyclic lactones including their use as an anti-inflammatory or anti-tumor agent. See also Szallasi et al. J. Biol. Chem. (1994), vol. 269, pp. 2118-2124; Zhang et al., Cancer Res. (1996), vol. 56, pp. 802-808; Hennings et al. Carcinogenesis (1987), vol. 8, pp. 1343-1346; Varterasian et al. Clin. Cancer Res. (2000), vol. 6, pp. 825-828; Mutter et al. Bioorganic & Med. Chem. (2000), vol. 8, pp. 1841-1860; each incorporated by reference herein in its entirety.

Of the bryostatin class of compounds, Bryostatin-1 is particularly interesting. It has been shown to activate PKC without tumor promotion. Further, its dose response curve is biphasic. In addition, Bryostatin-1 demonstrates differential regulation of PKC isoforms including PKC-α, PKC-δ and PKC-ε. Given this potential, Bryostatin-1 has undergone toxicity and safety studies in animals and humans, and is actively being investigated as an anti-cancer agent as an adjuvant with other potential anti-cancer agents.

Bryostatins as a class are thought to bind to the C1a site (one of the DAG binding sites) and cause translocation like a phorbol ester, but unlike the phorbol esters, does not promote tumors. Bryostatin-1 exhibits no toxicity at 20 μg/week, although the use of more than 35 μg/week may be associated with muscle pain. In rats, the acute LD₅₀ value for Bryostatin-1 is 68 μg/kg, and the acute LD₁₀ value is 45 μg/kg. Death in high doses results from hemorrhage.

Bryostatin crosses the blood-brain barrier and is slowly eliminated from the brain, exhibiting slow dissociation kinetics (t_{1/2}>12 hr). In the blood stream, bryostatin has a short half-life (t_{1/2}=1 hr). However, of an initial dose (via intravenous injection), 1% is in the blood at 100 hrs and is detectable in the blood for 14 days after a single injection. Bryostatin tends to accumulate in fatty tissues and is likely detoxified though glycolysation of OH groups and other well-known pathways for detoxification of xenobiotic compounds.

In one embodiment of the present disclosure, the macrocyclic lactone is a bryostatin. Bryostatins include, for example, Bryostatin-1, Bryostatin-2, Bryostatin-3, Bryostatin-4, Bryostatin-5, Bryostatin-6, Bryostatin-7, Bryostatin-8, Bryostatin-9, Bryostatin-10, Bryostatin-11, Bryostatin-12, Bryostatin-13, Bryostatin-14, Bryostatin-15, Bryostatin-16, Bryostatin-17, and Bryostatin-18.

In at least one embodiment, the bryostatin is Bryostatin-1 (shown below).

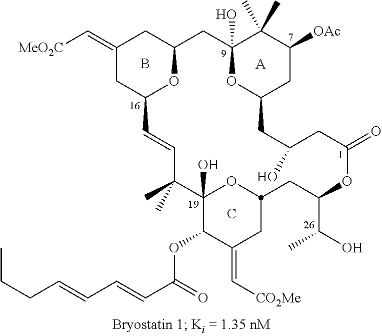

In another embodiment, the bryostatin is Bryostatin-2 (shown below; R=COC₇H₁₁, R′=H).

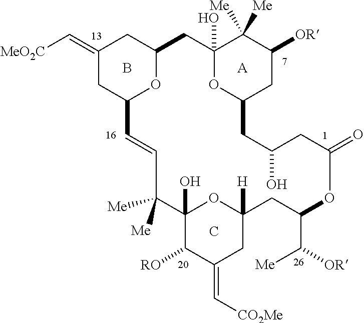

In one embodiment of the present disclosure, the macrocyclic lactone is a neristatin. In one embodiment, the neristatin is chosen from neristatin-1. In another embodiment, the macrocyclic lactone is chosen from macrocylic derivatives of cyclopropanated PUFAs such as, 24-octaheptacyclononacosan-25-one (cyclic DHA-CP6) (shown below).

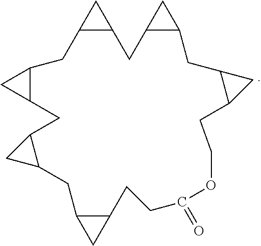 [SMILES: O=C1CCC2CC2CC2CC2CC2CC2CC2CC2CC2CC2CC2CC2CCO1]

In another embodiment, the macrocyclic lactone is a bryolog. Bryologs (analogs of bryostatin) are another class of PKC activators that are suitable for use in the present disclosure. Bryologs can be chemically synthesized or produced by certain bacteria. Different bryologs exist that modify, for example, the rings A, B, and C (see Bryostatin-1, figure shown above) as well as the various substituents. As a general overview, bryologs are considered less specific and less potent than bryostatin but are easier to prepare. It was found that the C-ring is important for binding to PKC while the A-ring is important for non-tumorigenesis. Further, the hydrophobic tail appears to be important for membrane binding.

Table 1 summarizes structural characteristics of several bryologs and demonstrates variability in their affinity for PKC (ranging from 0.25 nM to 10 μM). Structurally, they are all similar. While Bryostatin-1 has two pyran rings and one 6-membered cyclic acetal, in most bryologs one of the pyrans of Bryostatin-1 is replaced with a second 6-membered acetal ring. This modification reduces the stability of bryologs, relative to Bryostatin-1, for example, in both strong acid or base, but has little significance at physiological pH. Bryologs also have a lower molecular weight (ranging from about 600 g/mol to 755 g/mol), as compared to Bryostatin-1 (988), a property which facilitates transport across the blood-brain barrier.

TABLE 1
Bryologs.

| | PKC Affin | | |
|---|---|---|---|
| Name | (nM) | MW | Description |
| Bryostatin-1 | 1.35 | 988 | 2 pyran + 1 cyclic acetal + macrocycle |
| Analog 1 | 0.25 | 737 | 1 pyran + 2 cyclic acetal + macrocycle |
| Analog 2 | 6.50 | 723 | 1 pyran + 2 cyclic acetal + macrocycle |
| Analog 7a | — | 642 | 1 pyran + 2 cyclic acetals + macrocycle |
| Analog 7b | 297 | 711 | 1 pyran + 2 cyclic acetals + macrocycle |
| Analog 7c | 3.4 | 726 | 1 pyran + 2 cyclic acetals + macrocycle |
| Analog 7d | 10000 | 745 | 1 pyran + 2 cyclic acetals + |
| | | | macrocycle, acetylated |
| Analog 8 | 8.3 | 754 | 2 cyclic acetals + macrocycle |
| Analog 9 | 10000 | 599 | 2 cyclic acetals |

Analog 1 exhibits the highest affinity for PKC. Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649, each incorporated by reference herein in their entireties. Only Analog 1 exhibits a higher affinity for PKC than Bryostatin-1. Analog 2, which lacks the A ring of Bryostatin-1, is the simplest analog that maintains high affinity for PKC. In addition to the active bryologs, Analog 7d, which is acetylated at position 26, has virtually no affinity for PKC.

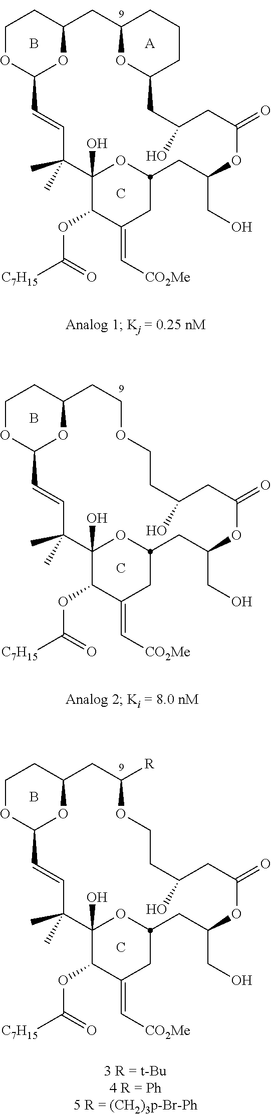

B-ring bryologs may also be used in the present disclosure. These synthetic bryologs have affinities in the low nanomolar range. Wender et aI., Org Lett. (2006), vol. 8, pp. 5299-5302, incorporated by reference herein in its entirety. B-ring bryologs have the advantage of being completely synthetic, and do not require purification from a natural source.

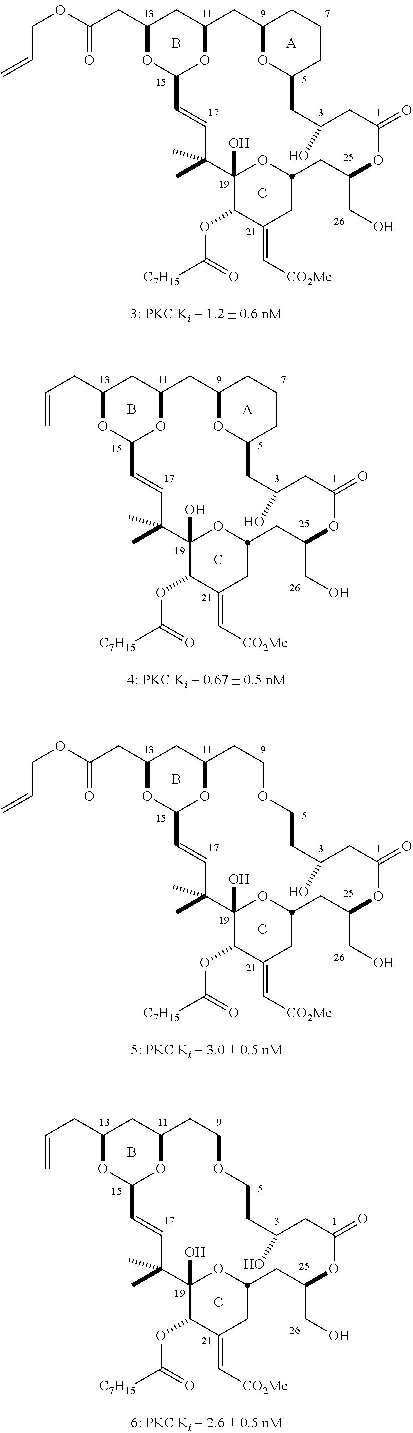

A third class of suitable bryostatin analogs are the A-ring bryologs. These bryologs have slightly lower affinity for PKC than Bryostatin-1 (6.5 nM, 2.3 nM, and 1.9 nM for bryologs 3, 4, and 5, respectively) and a lower molecular weight. A-ring substituents are important for non-tumorigenesis.

Bryostatin analogs are described, for example, in U.S. Pat. Nos. 6,624,189 and 7,256,286. Methods using macrocyclic lactones to improve cognitive ability are also described in U.S. Pat. No. 6,825,229 B2.

Another class of PKC activators is derivatives of diacylglycerols that bind to and activate PKC. See, e.g., Niedel et al., Proc. Natl. Acad. Sci. (1983), vol. 80, pp. 36-40; Mori et al., J. Biochem. (1982), vol. 91, pp. 427-431; Kaibuchi et al., J. Biol. Chem. (1983), vol. 258, pp. 6701-6704. Activation of PKC by diacylglycerols is transient, because they are rapidly metabolized by diacylglycerol kinase and lipase. Bishop et al. J. Biol. Chem. (1986), vol. 261, pp. 6993-7000; Chuang et al. Am. J. Physiol. (1993), vol. 265, pp. C927-C933; incorporated by reference herein in their entireties. The fatty acid substitution on the diacylglycerols derivatives determines the strength of activation. Diacylglycerols having an unsaturated fatty acid are most active. The stereoisomeric configuration is important; fatty acids with a 1,2-sn configuration are active while 2,3-sn-diacylglycerols and 1,3-diacylglycerols do not bind to PKC. Cis-unsaturated fatty acids may be synergistic with diacylglycerols. In at least one embodiment, the term “PKC activator” expressly excludes DAG or DAG derivatives.

Another class of PKC activators is isoprenoids. Farnesyl thiotriazole, for example, is a synthetic isoprenoid that activates PKC with a K_{d} of 2.5 μM. Farnesyl thiotriazole, for example, is equipotent with dioleoylglycerol, but does not possess hydrolyzable esters of fatty acids. Gilbert et al., Biochemistry (1995), vol. 34, pp. 3916-3920; incorporated by reference herein in its entirety. Farnesyl thiotriazole and related compounds represent a stable, persistent PKC activator. Because of its low molecular weight (305.5 g/mol) and absence of charged groups, farnesyl thiotriazole would be expected to readily cross the blood-brain barrier.

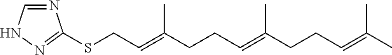 [SMILES: CC(C)=CCC/C(C)=C/CC/C(C)=C/CSc1nc[nH]n1]

Yet another class of activators includes octylindolactam V, gnidimacrin, and ingenol. Octylindolactam V is a non-phorbol protein kinase C activator related to teleocidin. The advantages of octylindolactam V (specifically the (-)-enantiomer) include greater metabolic stability, high potency (EC₅₀=29 nM) and low molecular weight that facilitates transport across the blood brain barrier. Fujiki et al. Adv. Cancer Res. (1987), vol. 49 pp. 223-264; Collins et al. Biochem. Biophys. Res. Commun. (1982), vol. 104, pp. 1159-4166, each incorporated by reference herein in its entirety.

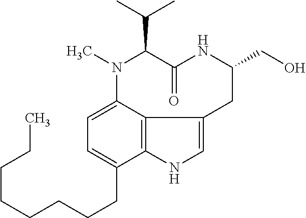 [SMILES: CCCCCCCCc1ccc2c3c(c[nH]c13)C[C@@H](CO)NC(=O)[C@H](C(C)C)N2C]

Gnidimacrin is a daphnane-type diterpene that displays potent antitumor activity at concentrations of 0.1 nM-1 nM against murine leukemias and solid tumors. It acts as a PKC activator at a concentration of 0.3 nM in K562 cells, and regulates cell cycle progression at the G1/S phase through the suppression of Cdc25A and subsequent inhibition of cyclin dependent kinase 2 (Cdk2) (100% inhibition achieved at 5 ng/ml). Gnidimacrin is a heterocyclic natural product similar to Bryostatin-1, but somewhat smaller (MW=774.9 g/mol).

Iripallidal is a bicyclic triterpenoid isolated from Iris pallida. Iripallidal displays anti-proliferative activity in a NCI 60 cell line screen with GI₅₀ (concentration required to inhibit growth by 50%) values from micromolar to nanomolar range. It binds to PKCα with high affinity (Kᵢ=75.6 nM). It induces phosphorylation of Erk1/2 in a RasGRP3-dependent manner. Its molecular weight is 486.7 g/mol. Iripallidal is about half the size of Bryostatin-1 and lacks charged groups.

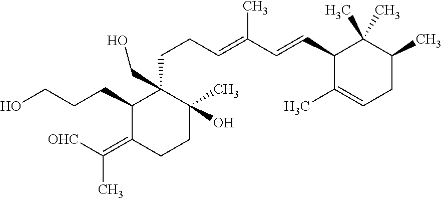

Ingenol is a diterpenoid related to phorbol but less toxic. It is derived from the milkweed plant Euphorbia peplus. Ingenol 3,20-dibenzoate, for example, competes with [3H] phorbol dibutyrate for binding to PKC (Kᵢ=240 nM). Winkler et al., J. Org. Chem. (1995), vol. 60, pp. 1381-1390, incorporated by reference herein. Ingenol-3-angelate exhibits antitumor activity against squamous cell carcinoma and melanoma when used topically. Ogbourne et al. Anticancer Drugs (2007), vol. 18, pp. 357-362, incorporated by reference herein.

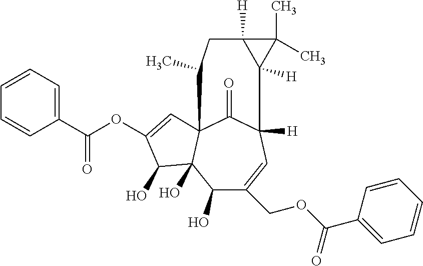 [SMILES: C[C@@H]1C[C@@H]2[C@H]([C@@H]3C=C(COC(=O)c4ccccc4)[C@@H](O)[C@]4(O)[C@@H](O)C(OC(=O)c5ccccc5)=C[C@@]14C3=O)C2(C)C]

Another class of PKC activators is napthalenesulfonamides, including N-(n-heptyl)-5-chloro-l-naphthalenesulfonamide (SC-10) and N-(6-phenylhexyl)-5-chloro-1-naphthalenesulfonamide. SC-10 activates PKC in a calcium-dependent manner, using a mechanism similar to that of phosphatidylserine. Ito et al., Biochemistry (1986), vol. 25, pp. 4179-4184, incorporated by reference herein. Naphthalenesulfonamides act by a different mechanism than bryostatin and may show a synergistic effect with bryostatin or member of another class of PKC activators. Structurally, naphthalenesulfonamides are similar to the calmodulin (CaM) antagonist W-7, but are reported to have no effect on CaM kinase.

Yet another class of PKC activators is diacylglycerol kinase inhibitors, which indirectly activate PKC. Examples of diacylglycerol kinase inhibitors include, but are not limited to, 6-(2-(4-[(4-fluorophenyl)phenylmethylene]-1-piperidinyl)ethyl)-7-methyl-5H-thiazolo[3,2-a]pyrimidin-5-one (R59022) and [3-[2-[4-(bis-(4-fluorophenyl)methylene]piperidin-1-yl)ethyl]-2,3-dihydro-2-thioxo-4(1H)-quinazolinone (R59949).

Still another class of PKC activators is growth factors, such as fibroblast growth factor 18 (FGF-18) and insulin growth factor, which function through the PKC pathway. FGF-18 expression is up-regulated in learning, and receptors for insulin growth factor have been implicated in learning. Activation of the PKC signaling pathway by these or other growth factors offers an additional potential means of activating PKC.

Another class of PKC activators is hormones and growth factor activators, including 4-methyl catechol derivatives like 4-methylcatechol acetic acid (MCBA) that stimulate the synthesis and/or activation of growth factors such as NGF and BDNF, which also activate PKC as well as convergent pathways responsible for synaptogenesis and/or neuritic branching.

Further example PKC activators include polyunsaturated fatty acids (“PUFAs”). These compounds are essential components of the nervous system and have numerous health benefits. In general, PUFAs increase membrane fluidity, rapidly oxidize to highly bioactive products, produce a variety of inflammatory and hormonal effects, and are rapidly degraded and metabolized. The inflammatory effects and rapid metabolism is likely the result of their active carbon-carbon double bonds. These compounds may be potent activators of PKC, most likely by binding the PS site.

In one embodiment, the PUFA is chosen from linoleic acid (shown below).

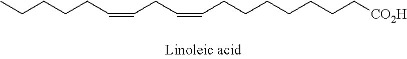 [SMILES: CCCCC/C=C\C/C=C\CCCCCCCC(=O)O]

Another class of PKC activators is PUFA and MUFA derivatives, and cyclopropanated derivatives in particular. Certain cyclopropanated PUFAs, such as DCPLA (i.e., linoleic acid with cyclopropane at both double bonds), may be able to selectively activate PKC-ε. See Journal of Biological Chemistry, 2009, 284(50): 34514-34521; see also U.S. Patent Application Publication No. 2010/0022645 A1 Like their parent molecules, PUFA derivatives are thought to activate PKC by binding to the PS site.

Cyclopropanated fatty acids exhibit low toxicity and are readily imported into the brain where they exhibit a long half-life (t_{1/2}). Conversion of the double bonds into cyclopropane rings prevents oxidation and metabolism to inflammatory byproducts and creates a more rigid U-shaped 3D structure that may result in greater PKC activation. Moreover, this U-shape may result in greater isoform specificity. For example, cyclopropanated fatty acids may exhibit potent and selective activation of PKC-ε.

The Simmons-Smith cyclopropanation reaction is an efficient way of converting double bonds to cyclopropane groups. This reaction, acting through a carbenoid intermediate, preserves the cis-stereochemistry of the parent molecule. Thus, the PKC-activating properties are increased while metabolism into other molecules like bioreactive eicosanoids, thromboxanes, or prostaglandins is prevented.

One class of PKC-activating fatty acids is Omega-3 PUFA derivatives. In one embodiment, the Omega-3 PUFA derivatives are chosen from cyclopropanated docosahexaenoic acid, cyclopropanated eicosapentaenoic acid, cyclopropanated rumelenic acid, cyclopropanated parinaric acid, and cyclopropanated linolenic acid (CP3 form shown below).

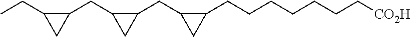 [SMILES: CCC1CC1CC1CC1CC1CC1CCCCCCCC(=O)O]

Another class of PKC-activating fatty acids is Omega-6 PUFA derivatives. In one embodiment, the Omega-6 PUFA derivatives are chosen from cyclopropanated linoleic acid (“DCPLA,” CP2 form shown below),

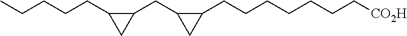 [SMILES: CCCCCC1CC1CC1CC1CCCCCCCC(=O)O]

cyclopropanated arachidonic acid, cyclopropanated eicosadienoic acid, cyclopropanated dihomo-gamma-linolenic acid, cyclopropanated docosadienoic acid, cyclopropanated adrenic acid, cyclopropanated calendic acid, cyclopropanated docosapentaenoic acid, cyclopropanated jacaric acid, cyclopropanated pinolenic acid, cyclopropanated podocarpic acid, cyclopropanated tetracosatetraenoic acid, and cyclopropanated tetracosapentaenoic acid.

Vernolic acid is a naturally occurring compound. However, it is an epoxyl derivative of linoleic acid and therefore, as used herein, is considered an Omega-6 PUFA derivative. In addition to vernolic acid, cyclopropanated vernolic acid (shown below) is an Omega-6 PUFA derivative.

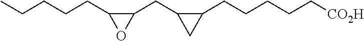 [SMILES: CCCCCC1OC1CC1CC1CCCCCC(=O)O]

Another class of PKC-activating fatty acids is Omega-9 PUFA derivatives. In one embodiment, the Omega-9 PUFA derivatives are chosen from cyclopropanated eicosenoic acid, cyclopropanated mead acid, cyclopropanated erucic acid, and cyclopropanated nervonic acid.

Yet another class of PKC-activating fatty acids is monounsaturated fatty acid (“MUFA”) derivatives. In one embodiment, the MUFA derivatives are chosen from cyclopropanated oleic acid (shown below),

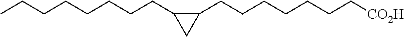 [SMILES: CCCCCCCCC1CC1CCCCCCCC(=O)O]

and cyclopropanated elaidic acid (shown below).

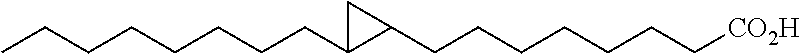 [SMILES: CCCCCCCCC1CC1CCCCCCCC(=O)O]

PKC-activating MUFA derivatives include epoxylated compounds such as trans-9,10-epoxystearic acid (shown below).

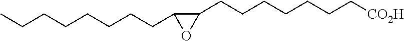 [SMILES: CCCCCCCCC1OC1CCCCCCCC(=O)O]

Another class of PKC-activating fatty acids is Omega-5 and Omega-7 PUFA derivatives. In one embodiment, the Omega-5 and Omega-7 PUFA derivatives are chosen from cyclopropanated rumenic acid, cyclopropanated alpha-elostearic acid, cyclopropanated catalpic acid, and cyclopropanated punicic acid.

Another class of PKC activators is fatty acid alcohols and derivatives thereof, such as cyclopropanated PUFA and MUFA fatty alcohols. It is thought that these alcohols activate PKC by binding to the PS site. These alcohols can be derived from different classes of fatty acids.

In one embodiment, the PKC-activating fatty alcohols are derived from Omega-3 PUFAs, Omega-6 PUFAs, Omega-9 PUFAs, and MUFAs, especially the fatty acids noted above. In one embodiment, the fatty alcohol is chosen from cyclopropanated linolenyl alcohol (CP3 form shown below),

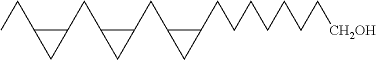 [SMILES: CCC1CC1CC1CC1CC1CC1CCCCCCCCO]

cyclopropanated linoleyl alcohol (CP2 form shown below),

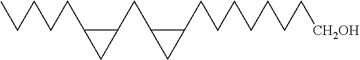 [SMILES: CCCCCC1CC1CC1CC1CCCCCCCCO]

cyclopropanated elaidic alcohol (shown below),

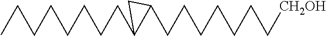 [SMILES: CCCCCCCCC1CC1CCCCCCCCO]

cyclopropanated DCPLA alcohol, and cyclopropanated oleyl alcohol.

Another class of PKC activators is fatty acid esters and derivatives thereof, such as cyclopropanated PUFA and MUFA fatty esters. In one embodiment, the cyclopropanated fatty esters are derived from Omega-3 PUFAs, Omega-6 PUFAs, Omega-9 PUFAs, MUFAs, Omega-5 PUFAs, and Omega-7 PUFAs. These compounds are thought to activate PKC through binding on the PS site. One advantage of such esters is that they are generally considered to be more stable that their free acid counterparts.

In one embodiment, the PKC-activating fatty acid esters derived from Omega-3 PUFAs are chosen from cyclopropanated eicosapentaenoic acid methyl ester (CP5 form shown below)

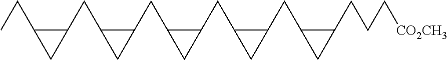 [SMILES: CCC1CC1CC1CC1CC1CC1CC1CC1CC1CC1CCCC(=O)OC]

and cyclopropanated linolenic acid methyl ester (CP3 form shown below).

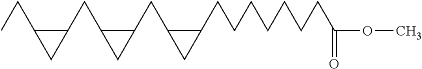 [SMILES: CCC1CC1CC1CC1CC1CC1CCCCCCCC(=O)OC]

In another embodiment, the Omega-3 PUFA esters are chosen from esters of DHA-CP6 and aliphatic and aromatic alcohols. In one embodiment, the ester is cyclopropanated docosahexaenoic acid methyl ester (CP6 form shown below).

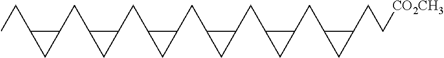 [SMILES: CCC1CC1CC1CC1CC1CC1CC1CC1CC1CC1CC1CC1CCC(=O)OC]

DHA-CP6, in fact, has been shown to be effective at a concentration of 10 nM. See, e.g., U.S. Patent Application Publication No. 2010/0022645.

In one embodiment, PKC-activating fatty esters derived from Omega-6 PUFAs are chosen from cyclopropanated arachidonic acid methyl ester (CP4 form shown below),

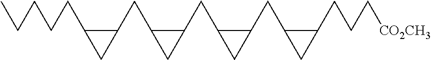 [SMILES: CCCCCC1CC1CC1CC1CC1CC1CC1CC1CCCC(=O)OC]

cyclopropanated vernolic acid methyl ester (CP1 form shown below), and

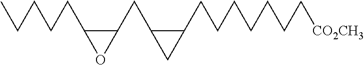 [SMILES: CCCCCC1OC1CC1CC1CCCCCCCC(=O)OC]

vernolic acid methyl ester (shown below).

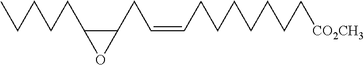 [SMILES: CCCCCC1OC1C/C=C\CCCCCCCC(=O)OC]

One particularly interesting class of esters are derivatives of DCPLA (CP6-linoleic acid). See, e.g., U.S. Provisional Patent Application No. 61/559,117 and applications claiming priority thereof. In one embodiment, the ester of DCPLA is an alkyl ester. The alkyl group of the DCPLA alkyl esters may be linear, branched, and/or cyclic. The alkyl groups may be saturated or unsaturated. When the alkyl group is an unsaturated cyclic alkyl group, the cyclic alkyl group may be aromatic. The alkyl group, in one embodiment, may be chosen from methyl, ethyl, propyl (e.g., isopropyl), and butyl (e.g., tert-butyl) esters. DCPLA in the methyl ester form (“DCPLA-ME”) is shown below.

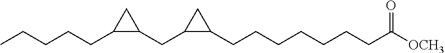 [SMILES: CCCCCC1CC1CC1CC1CCCCCCCC(=O)OC]

In another embodiment, the esters of DCPLA are derived from a benzyl alcohol (unsubstituted benzyl alcohol ester shown below). In yet another embodiment, the esters of DCPLA are derived from aromatic alcohols such as phenols used as antioxidants and natural phenols with pro-learning ability. Some specific examples include estradiol, butylated hydroxytoluene, resveratrol, polyhydroxylated aromatic compounds, and curcumin.

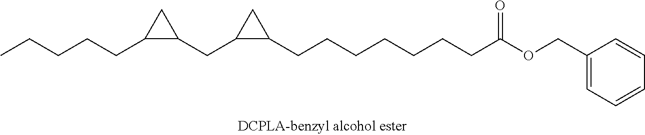 [SMILES: CCCCCC1CC1CC1CC1CCCCCCCC(=O)OCc1ccccc1]

Another class of PKC activators is fatty esters derived from cyclopropanated MUFAs. In one embodiment, the cyclopropanated MUFA ester is chosen from cyclopropanated elaidic acid methyl ester (shown below),

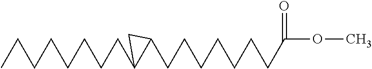 [SMILES: CCCCCCCCC1CC1CCCCCCCC(=O)OC]

and cyclopropanated oleic acid methyl ester (shown below).

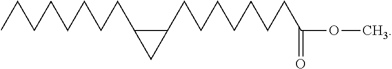 [SMILES: CCCCCCCCC1CC1CCCCCCCC(=O)OC]

Another class of PKC activators is sulfates and phosphates derived from PUFAs, MUFAs, and their derivatives. In one embodiment, the sulfate is chosen from DCPLA sulfate and DHA sulfate (CP6 form shown below).

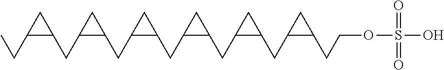 [SMILES: CCC1CC1CC1CC1CC1CC1CC1CC1CC1CC1CC1CC1CCOS(=O)(=O)O]

In one embodiment, the phosphate is chosen from DCPLA phosphate and DHA phosphate (CP6 form shown below).

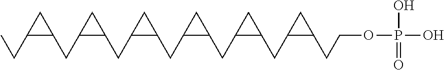 [SMILES: CCC1CC1CC1CC1CC1CC1CC1CC1CC1CC1CC1CC1CCOP(=O)(O)O]

In one embodiment the PKC activator is a macrocyclic lactone, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, or fatty acid esters.

### ApoE Regulates HDAC Nucleo-Cytoplasmic Shuttling Through LRP-1 and PKCε

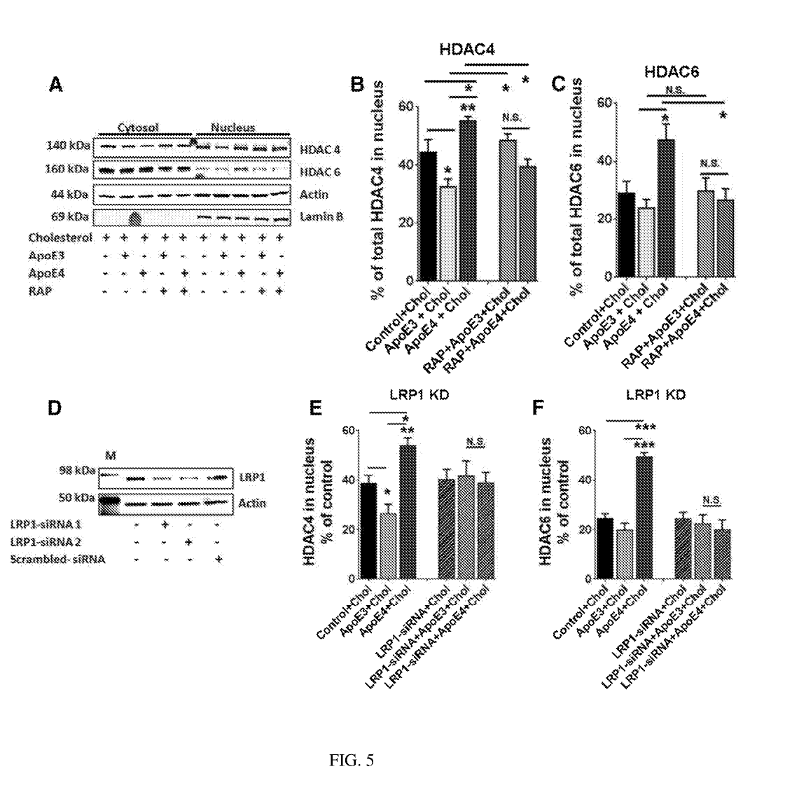

In the brain, ApoE is produced in astrocytes and transports cholesterol to neurons by interacting with ApoE receptors, such as members of the low-density lipoprotein receptor (LDLR) family and LRP-1. (Koryakina A, et al., Regulation of secretases by all-trans-retinoic acid, FEBS J, 276:2645-2655, 2009; Holtzman D M, et al., Apolipoprotein E and apolipoprotein E receptors: normal biology and roles in Alzheimer disease, Cold Spring Harb Perspect Med., 2:a006312, 2012; Liu C C, et al., 2013) Previous studies by the inventors showed that ApoE3 protects against ASPD induced synaptic damage thorough its interactions with LRP-1. (Sen A, et al., Apolipoprotein E3 (ApoE3) but not ApoE4 protects against synaptic loss through increased expression of protein kinase C epsilon, J. Biol. Chem., 287:15947-15958, 2012). Further supporting the role of LRP-1 in ApoE-mediated protection of neurons and ApoE mediated nuclear translocation of HDAC's is the observation that pretreating SH-SY5Y cells for 30 min with an ApoE receptor binding protein, such as RAP (100 nM; Migliorini et al., Allosteric modulation of ligand binding to low density lipoprotein receptor related protein by the receptor-associated protein requires critical lysine residues within its carboxyl-terminal domain, J. Biol. Chem., 278:17986-17992, 2003), followed by treatment of these cells with ApoE3+Chol or ApoE4+Chol for 24 h prevented nuclear translocation of HDACs in both treatment groups (FIG. 5A). In fact, blocking LRP-1 with RAP abolished the effects of ApoE3+Chol and ApoE4+Chol on nuclear translocation of HDAC4 (FIG. 5B) and HDAC6 (FIG. 5C).

Further evidence that LRP-1 regulates ApoE-mediated HDAC translocation, was obtained from a study involving LRP-1 siRNA to decrease LRP-1 levels in SH-SY5Y cells. As shown in FIG. 5D, gene silencing by LRP-1 siRNA 1 and LRP-1 siRNA 2 decreased cellular LRP-1 levels by ˜80% compared to control SH-SY5Y cells. ApoE4+Chol (38.8±4.5%) and ApoE3+Chol (41.6±6.3%) had no effect on HDAC4 translocation to the nucleus in LRP-1 downregulated cells (FIG. 5E) compared to cholesterol-treated control cells (40.2±4.3%). LRP-1 downregulation also prevented the ApoE4+Chol-mediated nuclear translocation of HDAC6 (FIG. 5F). These results indicate that ApoE acts via LRP-1 receptors to modulate nucleo-cytoplasmic shuttling of HDAC's.

ApoE-mediated HDAC nucleo-cytoplasmic shuttling is a determinant of neurodegenerative conditions. Previous studies by the inventors showed that the neuroprotective and synaptogenic effects of ApoE3 are mediated through PKCε and LRP-1, and in fact, ApoE3, but not ApoE4, induces PKCε transcription thus increasing PKCε levels in both control and ASPD-treated cells (Sen et al., 2012). Thus, the present inventors examined whether PKCε regulates ApoE-mediated HDAC nuclear translocation, by measuring the amount of PKCε, PKCα, and PKCδ mRNA in SH-SY5Y cells treated with cholesterol in the presence of ApoE3 or ApoE4.

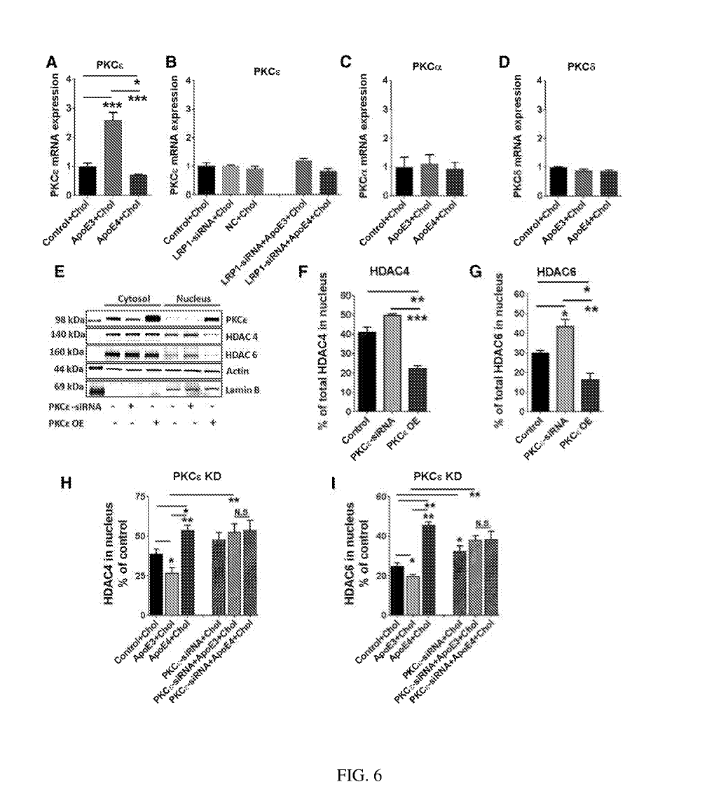

As illustrated in FIG. 6A, SH-SY5Y cells treated with ApoE3+Chol increased expression of PKCε mRNA by 2.5-fold compared with cholesterol-treated cells. In contrast, ApoE4+Chol reduced expression of PKCε mRNA levels. ApoE3+Chol, however, failed to increase the PKCε mRNA in LRP-1 downregulated cells (FIG. 6B), and no change in the transcript levels of either PKCα or PKCδ were observed in ApoE3+Chol or ApoE4+Chol treated cells (FIG. 6C, D).

PKCε overexpression (FIG. 6E) further reduced nuclear HDAC4 by 1.83-fold compared with control cells (40.9±2.8% vs 22.3±1.6%; t test, p<0.005; FIG. 6F). Overexpression of PKCε also reduced nuclear HDAC6 levels by 54% compared with control cells (29.9±1.4% vs 16.3±3.2%; t test, p<0.005; FIG. 6G). Inhibiting cellular PKCε expression (PKCε knock-downs), however, had no effect on nuclear HDAC4 levels but increased HDAC6 levels in the nucleus by 1.4-fold compared with control cells (43.3±3.8% vs 29.9±1.4%; t test, p<0.05; FIG. 6G). The above data indicated that while PKCε is involved in nuclear retention of HDAC4, PKCε is required for retention of HDAC6 in the cytosol.

PKCε gene silencing studies provided further proof that ApoE mediated nucleo-cytoplasmic shuttling of HDAC's is regulated by PKCε. A PKCε-siRNA was introduced into SH-SY5Y cells. These PKCε knock-down cells were treated with ApoE3+Chol.

As illustrated in FIG. 6H, HDAC4 nuclear export by ApoE3+Chol (52.6±5.4%; t test, p<0.01 vs 26.6±3.8% in normal cells) was abolished in PKCε knock-downs. Inhibition of cellular PKCε synthesis by gene silencing also abolished the effect of ApoE3+Chol on HDAC6 levels in the nucleus (37.9±2.1%; t test, p<0.002 vs 19.7±1.1% in normal cells; FIG. 6I).

Bryostatin and BR-122, an analog of bryostatin are activators of PKC. BR-122 increased PKCε levels in primary neurons while a single intravenous injection of bryostatin was observed to activate PKCε expression and increase PKCε levels in the brain of mice. See FIGS. 10 and 11, respectively.

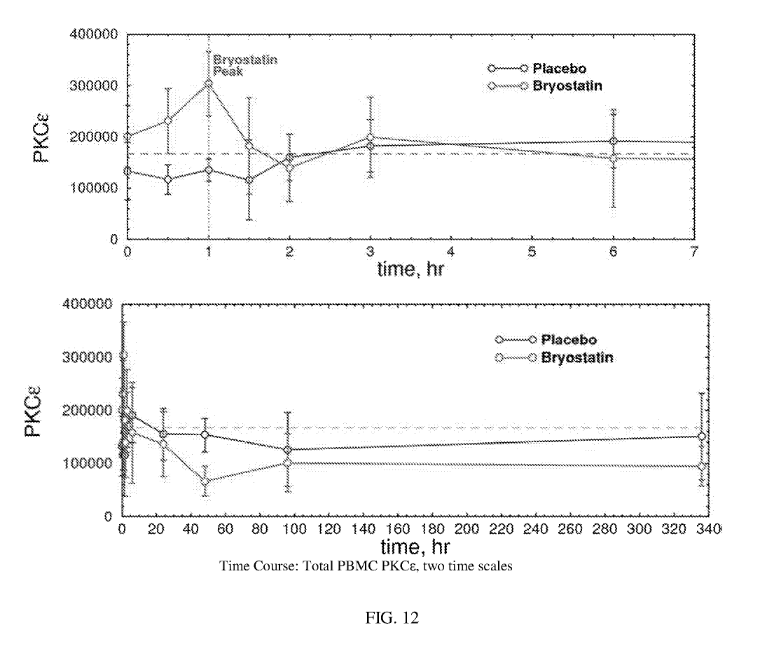

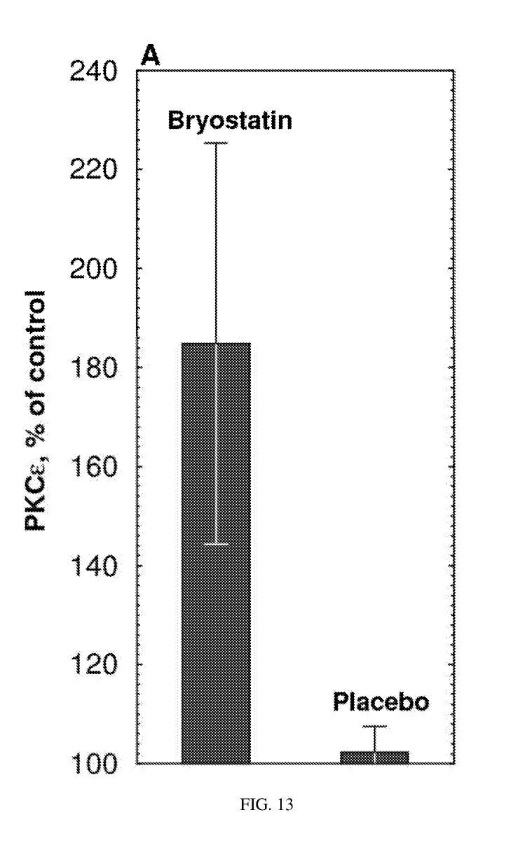

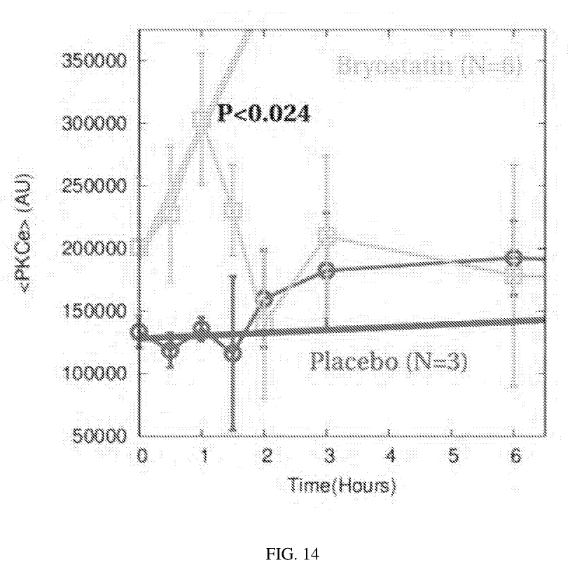

From Phase IIa clinical trials, the data showed that administration of bryostatin increases the synthesis of PKCε. As illustrated in FIGS. 12 and 13, PKCε levels are highest at 1 h post administration of bryostatin. Specifically, as illustrated in FIG. 14, increases in PKCε levels correlate with the physiological increase bryostatin levels in subjects receiving bryostatin injections.

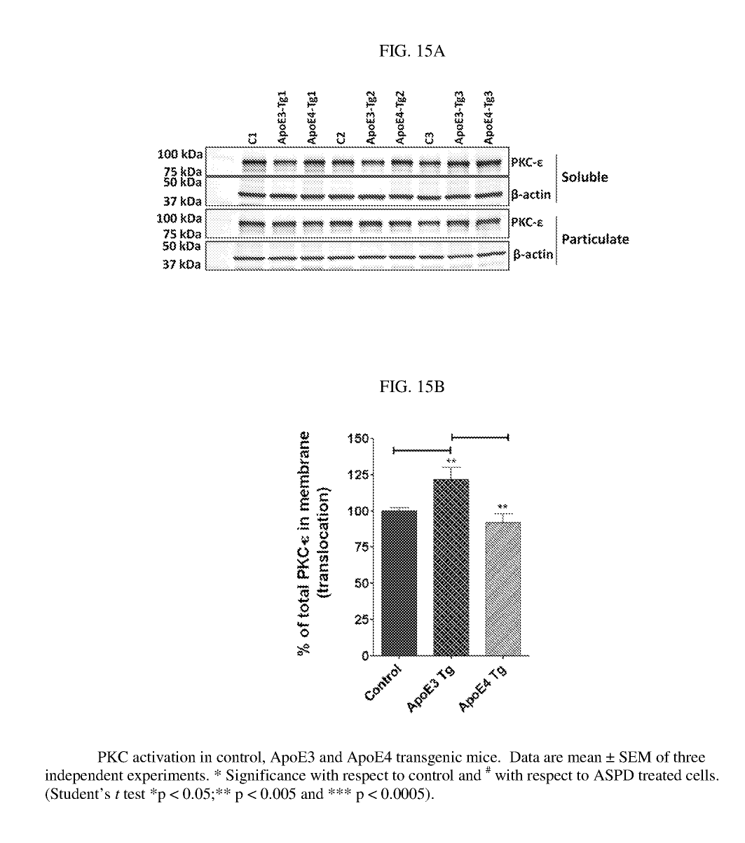

Further studies showed that in mice expressing hApoE3, PKC was constitutively activated as shown by the increased percentage of total PKC. See FIG. 15. In contrast total PKC levels were much lower in mice expressing hApoE4.

### ApoE3 Regulates BDNF Expression—Implications in Reducing Neurodegenerative Pathology

BDNF is known to exert a neuroprotective role. In fact, recent studies have shown additive effects for ApoE and BDNF in memory-related disorders (Kauppi et al., Additive genetic effect of APOE and BDNF on hippocampus activity, Neuroimage, 89:306-313, 2014; Lim et al., APOE and BDNF polymorphisms moderate amyloid beta-related cognitive decline in preclinical Alzheimer' s disease, Mol Psychiatry, 2014).

As described above, ApoE3 induces PKCε expression in rat primary neurons (Sen et al., 2012) and in human SH-SY5Y cells. PKCε, however, is known to regulate BDNF expression (Hongpaisan et al., 2011; Lim and Alkon, 2012; Hongpaisan et al., 2013; and Neumann et al., 2015). From these findings, the present inventors hypothesized that ApoE3 may be involved in the regulation of BDNF expression.

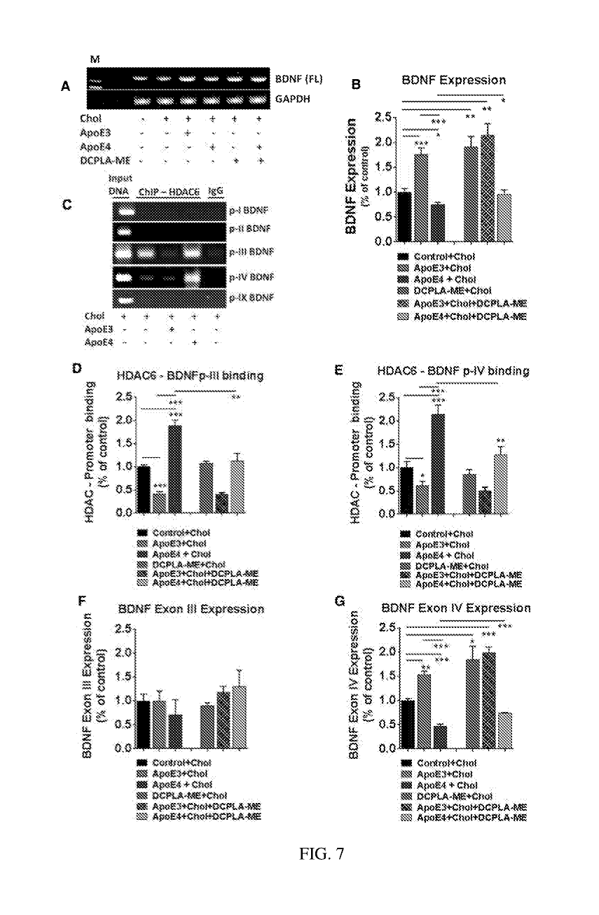

To delineate the link between ApoE isoforms, PKCε, HDACs and BDNF, the inventors measured mRNA expression of BDNF using primers in SH-SY5Y cells by semiquantitative RT-PCR (full-length; FIG. 7A) and qRT-PCR (FIG. 7B). SH-SY5Y cells treated with cholesterol alone showed no change in BDNF expression (FIG. 7A). Cells treated with ApoE3+Chol, however, showed increased BDNF expression (1.76_0.13; t test, p_0.001) while ApoE4+Chol downregulated BDNF expression (0.74_0.05; p_0.02; FIG. 7B).

Further support that PKCε is as effective as ApoE3 in regulating BDNF expression is provided by the observation that DCPLA-ME, a PKCε specific activator (Nelson et al., 2009; Sen et al., 2012), upregulates BDNF expression in cholesterol treated cells (1.91±0.21-fold). In fact, BDNF expression was upregulated by DCPLA-ME to nearly the same extent as ApoE3.

Further studies by the inventors showed that DCPLA-ME prevented BDNF downregulation in cells treated with ApoE4+Chol+DCPLA-ME (0.95±0.1; p<0.05). As a result, the present disclosure uses PKC activators as therapeutics for reversing the effects of or treating a neurodegenerative condition associated with ApoE regulation.

Further experiments in which cells were treated with a combination of ApoE3 and DCPLA-ME showed that the BDNF expression promoting effects ApoE3 and DCPLA-ME were not additive.

Previous studies have shown that patients with the ApoE4 allele have reduced levels of BDNF (Maioli et al., 2012; Alvarez et al., 2014). However, the mechanism by which ApoE4 downregulates BDNF expression in neurons was not known. The present inventors investigated whether ApoE4 downregulates BDNF expression in neurons by inducing HDAC6-BDNF PIII/PIV association. To elucidate the role of BDNF promoters in regulation of BDNF expression by ApoE, chromosome immunoprecipitation was performed using HDAC4, HDAC6 or IgG (as a Control) from SH-SY5Y cells treated with cholesterol, ApoE isoforms, and DCPLA-ME. A fixed fraction (2%) of the total unprecipitated DNA (used a positive control, used to normalize the results) or 100% of the HDAC-or IgG-immunoprecipitated DNA were amplified by PCR against BDNF promoters PI, PII, PIII, PIV and PIX. HDAC4 and HDAC6 (FIG. 7C) showed no association to PI, PII or PIX. ApoE3+Chol reduced HDAC6-PIII association (0.41±0.05; p<0.001) and ApoE4+Chol increased it (1.9±0.13; p<0.001) compared with cholesterol-only treated cells (1.0±0.04; F_{(5,24)}=51.8; ANOVA, p<0.0001; FIG. 7D). ApoE4+Chol also increased HDAC6-PIV association and ApoE3+Chol reduced it. (Cholesterol-only=1±0.13; ApoE3+Chol=0.61±0.10, p<0.05; ApoE4+Chol=2.1±0.20; p<0.001; F_{(5,30)}=18.6; ANOVA, p<0.0001; FIG. 7E). DCPLA-ME blocked the ApoE4+Chol induced increase in HDAC6-PIII and HDAC6-PIV association, but had no effect on ApoE3 (FIG. 7D,E). This is consistent with ApoE4 either directly or indirectly inhibiting PKCε synthesis, and PKCε inhibits HDAC6 transport to the nucleus where it can bind the promoter.

HDAC4 also co-immunoprecipitated with BDNF PIII and PIV, but no significant difference was noticed in binding among treated groups (HDAC4-PIII=F_{(5,24)}=1.1; ANOVA, p<0.4; HDAC4-PIV=F_{(5,24)}=0.98; ANOVA, p<0.46; results not shown). From this, PKCε activity blocks HDAC4 transport to the nucleus, thereby preventing HDAC4 from binding to BDNF promoters III and IV, whereas HDAC4 does not bind the promoters directly, but binds indirectly via transcription factors such as MEF2C and MEF2D. ApoE4 does not induce PKCε, thereby allowing HDAC to enter the nucleus, bind (indirectly) to the BDNF promoter, and thereby repress BDNF expression.

To determine the effect of HDAC6-PIII/PIV association on BDNF expression, the expression of BDNF-exon III and IV by qRT-PCR was analyzed. BDNF-exon IV expression was increased by ApoE3+Chol and decreased by ApoE4+Chol (1.54±0.07-fold; p<0.0027 and 0.47±0.04-fold; p<0.0005, respectively; FIG. 7G). BDNF-exon III expression showed a trend toward lower expression when ApoE4 was added, but it was not statistically significant (FIG. 7F). DCPLA-ME increased expression of exon IV by approximately the same percentage in all three treatments (F_{(5,21)}=40.0; ANOVA, p<0.0001; DCPLA-ME+Chol=1.85±0.27-fold, p<0.035; ApoE3+Chol+DCPLA-ME=1.98±0.13, p<0.05 vs ApoE3+Chol; ApoE4+Chol+DCPLA-ME=0.74±0.01, p<0.0005 vs ApoE4+Chol; FIG. 7G). ApoE4 induces an interaction between HDAC6 and BDNF-PIV that leads to reduced BDNF expression. PKC activators increased BDNF exon IV expression but had little effect on exon III expression, indicating that exon IV is responsive to PKC but exon III is not.

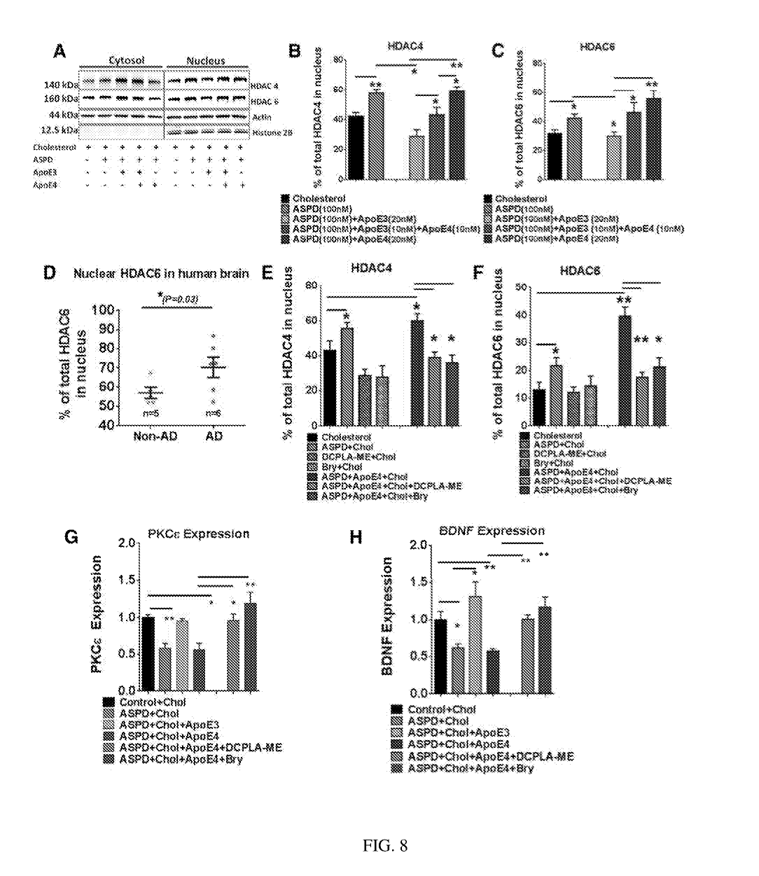

ApoE4 increases ASPD-induced nuclear translocation of HDACs. Immunoblots were used to examine the effect of ASPDs, a neurotoxic form of AP present in the AD brain, on HDAC4 and HDAC6 nuclear import. Human SH-SY5Y cells were treated with cholesterol and ApoE3 or ApoE4 in the presence or absence of ASPDs. ASPDs increased the import of both HDAC4 and HDAC6. Addition of ApoE3+Chol significantly reduced the percentage of HDAC4 and HDAC6 staining in the nucleus (−48.4±9.7%; p<0.026 and −29.3±6.9%; p<0.01; FIG. 8A). In contrast, ApoE4+Chol had no effect on the nuclear import of HDAC4 (+5.7±13% change; FIG. 8B) and increased the import of HDAC6 (+39.4±16.9% change, p<0.04) in the presence of ASPD (FIG. 8C). Cells treated with both ApoE3 and ApoE4 (10 nM) and cholesterol showed intermediate levels of nuclear HDAC4 and HDAC6 (HDAC4=F_{(2,8)}=14.2; ANOVA, p<0.005 and HDAC6=F_{(2,8)}=7.15; ANOVA; p<0.03; FIG. 8B,C). ApoE3 inhibits the effect of ASPDs on nuclear translocation of HDACs, but ApoE4 does not. The amount of nuclear HDAC6 from human brain hippocampus was also analyzed. Autopsy-confirmed AD cases showed increased nuclear HDAC6 compared with age-matched controls (AD=70.4±5.3% and non-AD=56.9±2.9; p<0.05; FIG. 8D).

PKCε activation inhibits ApoE4-induced nuclear translocation of HDACs. To investigate the effect of PKCε activation on ApoE4+ASPD induced nuclear translocation of HDAC4 and HDAC6, SH-SY5Y cells for 24 h were treated with combinations of ASPDs, cholesterol, and ApoE4 and were measured HDAC4 and HDAC6 levels in the cytosol and nucleus by immunoblotting. ASPD and ApoE4 had a synergistic effect on HDAC6 (Chol=12.9±3.0%; ASPD+APOE4+Chol=39.7±3.3%, p<0.005; ASPD+Chol=21.7±2.8%, p<0.05), but not HDAC4 (FIG. 8E,F). PKCε activation by DCPLA-ME or bryostatin 1 reduced the nuclear import of HDAC4 by 35±6.9% (p<0.015) and 40±8.4% (p<0.013), respectively (F_{(6,14)}=11.6; ANOVA, p<0.0001; FIG. 8E). Similar reductions in nuclear HDAC6 also occurred with DCPLA-ME (56±5.9% reduction, p<0.005) or bryostatin-1 (46±9.2% reduction, p<0.05; FIG. 8F). In each case, regardless of whether ASPDs, ApoE4, or a combination of both was used, PKC activation reduced HDAC import to normal or below normal levels.

Finally, the levels of PKCε and BDNF mRNA expression (using qRT-PCR) were measured in these ASPD-treated cells. PKCε levels were downregulated by ASPD+Chol (0.58±0.06-fold; p<0.005). ApoE3 restored PKCε expression to normal, but ApoE4 did not (0.56±0.09-fold; p<0.02). DCPLA-ME and bryostatin-1 counteracted the ASPD+ApoE4-mediated loss of PKCε (0.95±0.09-fold and 1.19±0.14%, respectively; FIG. 8G). BDNF was also downregulated by ASPD+Chol (0.62±0.05-fold; p<0.04; FIG. 8H). The addition of ApoE4 did not increase the effect of ASPD. ApoE3 prevented BDNF downregulation by ASPDs (1.32±0.19-fold; p<0.025 vs ASPD+Chol). PKCε activation also prevented BDNF loss in these cells (FIG. 8H). PKCε activation reverses the ApoE4-mediated nuclear translocation of HDAC, thereby restoring BDNF synthesis to normal levels. Here, bryostatin and DCPLA-ME corrected the deficiency of PKCε in those instances of the stimulated neurodegenerative disease state, e.g., ASPD+Chol and ASPD+Chol+ApoE4. This further evidences the use of PKC activators as therapeutics for reversing the effects of or treating a neurodegenerative condition associated with ApoE regulation.

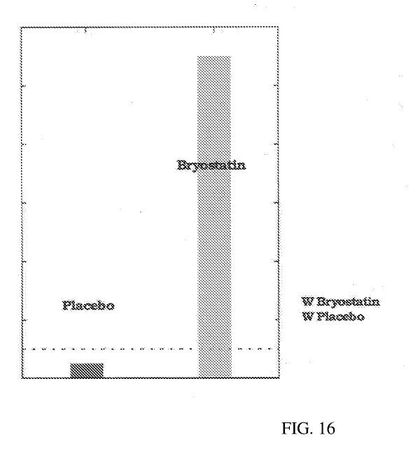

FIG. 16 illustrates cognitive improvement in subject receiving the PKC activator bryostatin, As illustrated, the mini-mental state examination score (MMSE) for bryostatin treated subjects was at least 2-fold greater than the MMSE score for placebo treated subjects. These results further illustrate that bryostatin can cross the blood-brain barrier after intravenous administration.

The one or more PKC activator or combination of one PKC activator may be administered to a patient/subject in need thereof by conventional methods such as oral, parenteral, transmucosal, intranasal, inhalation, or transdermal administration. Parenteral administration includes intravenous, intra-arteriolar, intramuscular, intradermal, subcutaneous, intraperitoneal, intraventricular, intrathecal, ICV, intracisternal injections or infusions and intracranial administration.

The present disclosure relates to compositions comprising one or more protein kinase C activator or combinations thereof and a carrier. The present disclosure further relates to a composition of at least one protein kinase C activator and a carrier, and a composition of at least one combination and a carrier, wherein the two compositions are administered together to a patient in need thereof. In one embodiment, the composition of at least one protein kinase C activator may be administered before or after the administration of the composition of the combination to a patient in need thereof.

The formulations of the compositions described herein may be prepared by any suitable method known in the art. In general, such preparatory methods include bringing at least one of active ingredients into association with a carrier. If necessary or desirable, the resultant product can be shaped or packaged into a desired single- or multi-dose unit.

Although the descriptions of compositions provided herein are principally directed to compositions suitable for ethical administration to humans, it will be understood by a skilled artisan that such compositions are generally suitable for administration to animals of all sorts. Modification of pharmaceutical compositions suitable for administration to humans or to render the compositions suitable for administration to various animals is well understood, and the ordinarily skilled veterinary pharmacologist can design and perform such modification with merely ordinary, if any, experimentation. Subjects to which administration of the compositions of the disclosure is contemplated include, but are not limited to, humans and other primates, and other mammals.

As discussed herein, carriers include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the compositions of the disclosure are generally known in the art and may be described, for example, in Remington's Pharmaceutical Sciences, Genaro, ed., Mack Publishing Co., Easton, Pa., 1985, and Remington's Pharmaceutical Sciences, 20^{th} Ed., Mack Publishing Co. 2000, both incorporated by reference herein.

In one embodiment, the carrier is an aqueous or hydrophilic carrier. In a further embodiment, the carrier can be water, saline, or dimethylsulfoxide. In another embodiment, the carrier is a hydrophobic carrier. Hydrophobic carriers include inclusion complexes, dispersions (such as micelles, microemulsions, and emulsions), and liposomes. Exemplary hydrophobic carriers include inclusion complexes, micelles, and liposomes. See, e.g., Remington's: The Science and Practice of Pharmacy 20th ed., ed. Gennaro, Lippincott: Philadelphia, Pa. 2003, incorporated by reference herein. In addition, other compounds may be included either in the hydrophobic carrier or the solution, e.g., to stabilize the formulation.

The compositions disclosed herein may be administrated to a patient in need thereof by any suitable route including oral, parenteral, transmucosal, intranasal, inhalation, or transdermal routes. Parenteral routes include intravenous, intra-arteriolar, intramuscular, intradermal, subcutaneous, intraperitoneal, intraventricular, intrathecal, and intracranial administration. A suitable route of administration may be chosen to permit crossing the blood-brain barrier. See e.g., J. Lipid Res. (2001) vol. 42, pp. 678-685, incorporated by reference herein.

In one embodiment, the compositions described herein may be formulated in oral dosage forms. For oral administration, the composition may take the form of a tablet or capsule prepared by conventional means with, for example, carriers such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods generally known in the art.

In another embodiment, the compositions herein are formulated into a liquid preparation. Such preparations may take the form of, for example, solutions, syrups or suspensions, or they may be presented as a dry product for constitution with water or other suitable vehicle before use. Such liquid preparations may be prepared by conventional means with, for examples, pharmaceutically acceptable carriers such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl p-hydroxybenzoates, or sorbic acid). The preparations may also comprise buffer salts, flavoring, coloring, and sweetening agents as appropriate. In one embodiment, the liquid preparation is for oral administration.

In another embodiment of the present disclosure, the compositions herein may be formulated for parenteral administration such as bolus injection or continuous infusion. Formulations for injection may be presented in unit dosage form, e.g., in ampoules, or in multi-dose containers, with an added preservative. The compositions may take such forms as suspensions, solutions, dispersions, or emulsions in oily or aqueous vehicles, and may contain formulatory agents such as suspending, stabilizing, and/or dispersing agents.

In another embodiment, the compositions herein may be formulated as depot preparations. Such formulations may be administered by implantation (for example, subcutaneously or intramuscularly) or by intramuscular injection. For example, the compositions may be formulated with a suitable polymeric or hydrophobic material (for example, as an emulsion in an acceptable oil) or ion exchange resin, or as a sparingly soluble derivative, for example, as a sparingly soluble salt.

In another embodiment, at least one PKC activator or combination thereof is delivered in a vesicle, such as a micelle, liposome, or an artificial low-density lipoprotein (LDL) particle. See, e.g., U.S. Pat. No. 7,682,627.

In a further embodiment, the doses for administration to a patient in need thereof may suitably be prepared so as to deliver from about 1 mg to about 10 g, such as from about 5 mg to about 5 g, from about 50 mg to about 2 g, from about 100 mg to about 1.5 g, from about 150 mg to about 1 g, or from about 250 mg to about 500 mg of at least one PKC activator or combination thereof.

In one embodiment, at least one PKC activator or combination thereof may be present in the composition in an amount ranging from about 0.01% to about 100%, from about 0.1% to about 90%, from about 0.1% to about 60%, from about 0.1% to about 30% by weight, or from about 1% to about 10% by weight of the final formulation. In another embodiment, at least one PKC activator or combination thereof may be present in the composition in an amount ranging from about 0.01% to about 100%, from about 0.1% to about 95%, from about 1% to about 90%, from about 5% to about 85%, from about 10% to about 80%, and from about 25% to about 75%.

The present disclosure further relates to kits that may be utilized for administering to a subject one or more PKC activator or combination thereof separately or combined in a single composition.

The kits may comprise devices for storage and/or administration. For example, the kits may comprise syringe(s), needle(s), needle-less injection device(s), sterile pad(s), swab(s), vial(s), ampoule(s), cartridge(s), bottle(s), and the like. The storage and/or administration devices may be graduated to allow, for example, measuring volumes. In one embodiment, the kit comprises at least one PKC activator in a container separate from other components in the system. In another embodiment, the kit comprises a means to combine at least one PKC activator and at least one combination separately. In yet another embodiment, the kit comprises a container comprising at least one PKC activator and a combination thereof.

The kits may also comprise one or more anesthetics, such as local anesthetics. In one embodiment, the anesthetics are in a ready-to-use formulation, for example an injectable formulation (optionally in one or more pre-loaded syringes), or a formulation that may be applied topically. Topical formulations of anesthetics may be in the form of an anesthetic applied to a pad, swab, towelette, disposable napkin, cloth, patch, bandage, gauze, cotton ball, Q-tip™, ointment, cream, gel, paste, liquid, or any other topically applied formulation. Anesthetics for use with the present disclosure may include, but are not limited to lidocaine, marcaine, cocaine, and xylocaine.

The kits may also contain instructions relating to the use of at least one PKC activator or a combination thereof. In another embodiment, the kit may contain instructions relating to procedures for mixing, diluting, or preparing formulations of at least one PKC activator or a combination thereof. The instructions may also contain directions for properly diluting a formulation of at least one PKC activator or a combination thereof in order to obtain a desired pH or range of pHs and/or a desired specific activity and/or protein concentration after mixing but prior to administration. The instructions may also contain dosing information. The instructions may also contain material directed to methods for selecting subjects for treatment with at least one PKC activator or a combination thereof.

The PKC activator can be formulated, alone in suitable dosage unit formulations containing conventional non-toxic pharmaceutically acceptable carriers, adjuvants and vehicles appropriate for each route of administration. Pharmaceutical compositions may further comprise other therapeutically active compounds which are approved for the treatment of neurodegenerative diseases or to reduce the risk of developing a neurodegenerative disorder.

Appropriate dosages of the PKC activator will generally be about 0.001 to 100 μg/m²/week which can be administered in single or multiple doses. For example, the dosage level will be about 0.01 to about 25 μg/m²/week; about 1 to about 20 μg/m²/week, about 5 to about 20 μg/m²/week, or about 10 to about 20 μg/m²/week. A suitable dosage may be about 5 μg/m²/week, about 10 μg/m²/week, about 15 μg/m²/week, or about 20 μg/m²/week.

For oral administration, the compositions are preferably provided in the form of tablets containing about 1 to 1000 micrograms of the active ingredient, particularly about 1, 5, 10, 15, 20, 25, 50, 75, 100, 150, 200, 250, 300, 400, 500, 600, 750, 800, 900, and 1000 micrograms of an active ingredient such as a PKC activator.

The pharmaceutical compositions according to the invention can be administered more than once a week, for example, using a regimen that comprises administering the composition 2, 3, 4, or 5 times a week. For certain neurodegenerative conditions, the pharmaceutical composition is administered daily, for example, once per day, twice per day, or at regular intervals of time such as weekly or every other week, two weeks, three weeks or four weeks.

It will be understood, however, that the specific dose and frequency of dosage for any particular patient may be varied and will depend upon a variety of factors including the activity of the compound formulated, the metabolic stability and length of action of that compound, the age, body weight, general health, sex, diet, mode and time of administration, rate of excretion, drug combinations used, and the severity of the particular neurodegenerative condition.

All of the references, patents and printed publications mentioned in the instant disclosure are hereby incorporated by reference in their entirety into this application.

The following examples are provided by way of illustration to further describe certain preferred embodiments of the invention, and are not intended to be limiting of the present invention.

### EXAMPLES

a. Materials

Cell culture media was purchased from Invitrogen (F12K, neurobasal, and B27) and K.D. Medical (MEM). Bryostatin-1 was purchased from Biomol International. DCPLA methyl ester (DCPLA-ME) was synthesized using a method described previously (Nelson et al., Neuroprotective versus tumorigenic protein kinase C activators, Trends Biochem Sci., 34:136-145, 2009). ApoE3 (rh-ApoE3), ApoE4 (rh-ApoE3), and other reagents were purchased from Sigma-Aldrich. Aβ₁₋₄₂ was purchased from Anaspec. Recombinant human receptor-associated protein (RAP) was purchased from Molecular Innovations and primary antibodies against acetylated histone 3, histone 3, β-actin, lamin B, and PKCε were purchased from Santa Cruz Biotechnology. Primary antibodies against HDAC1, HDAC2, HDAC3, HDAC4, HDAC5, and HDAC6 were purchased from Cell Signaling Technology, while all secondary antibodies were from Jackson ImmunoResearch Laboratories.

b. Synthesis of ASPD's

ASPDs were prepared as previously described (Noguchi et al., Isolation and characterization of patient-derived, toxic, high mass amyloid beta-protein (Abeta) assembly from Alzheimer disease brains, J. Biol. Chem., 284:32895-32905, 2009; and Sen et al., 2012, supra). Briefly, Aβ₁₋₄₂ was dissolved in 1,1,1,3,3,3-hexafluoro-2-propanol and incubated overnight at 4° C. The solution was then warmed to 37° C. and maintained at this temperature for 3 h. The dissolved Aβ₃₁₋₄₂ was lyophilized to obtain 40 nmol/tube. The lyophilized Aβ was dissolved in PBS without Ca²⁺ or Mg²⁺ to obtain a solution in which the concentration Aβ is <50 μM. This PBS solution is rotated for 14 h at 4° C. and the resulting ASPD solution was purified using a 100 kDa molecular weight cutoff filter (Amicon Ultra; Millipore).

c. Cell Culture and Treatment.

Human SH-SY5Y neuroblastoma cells (Sigma-Aldrich), were cultured in 45% F12K, 45% MEM, and 10% FBS. Cells were treated with cholesterol, ASPD, ApoE3/ApoE4+cholesterol, or PKC activators for 24 h. Cholesterol was dissolved in ethanol. ApoE (20 nM) and cholesterol (100 μM) were mixed separately into the cultures. To block the ApoE receptors, cells were treated with RAP for 30 min before adding ApoE. Human primary neurons (ScienCell Research Laboratories) were plated on poly-L-lysine-coated plates and were maintained in neuronal medium (ScienCell Research Laboratories) supplemented with the neuronal growth supplements (ScienCell Research Laboratories). Half of the culture medium was changed every 3 d and fresh activators were added with every medium change to maintain viable neurons.

d. Transgenic Mice

C57BL/6 mice for ApoE target replacement were obtained from Taconic Farms. The endogenous murine ApoE gene was replaced with human alleles of ApoE3 (B6.129P2-Apoe^{tm2(APOE*3)Mae}N8) or ApoE4 (B6.129P2-Apoe^{tm3(APOE*4)Mae}N8). All experiments were performed on age-matched male animals. All animals were housed in a barrier facility, provided food and water ad libitum, and maintained following the National Institutes of Health's Guide for the Care and Use of Laboratory Animals.

e. Human Brain Tissue

Fresh frozen human brain tissue was obtained from Harvard Brain Tissue Resource Center (McLean Hospital, Boston, Mass.) after approval for the study from Francine M. Benes (Table 2). Informed consent was obtained from all patients or legal representatives. The pathological diagnosis of AD was conducted according to the Consortium to Establish a Registry for Alzheimer's Disease (CERAD). The study was performed in accordance with the Code of Ethics of the World Medical Association (Declaration of Helsinki) for experiments involving humans

TABLE 2
Patient ID, Braak stage, and age of human brain tissue

| Patient ID | Sex | Age | Average age ± SD | Autopsy diagnosis |
|---|---|---|---|---|
| AN02930 | M | 80 | 77.5 ± 12.34 | AD Braak 3 |
| AN14554 | F | 61 | | AD Braak 6 |
| AN17726 | M | 72 | | AD Braak 2 |
| AN06468 | M | 98 | | AD Braak 4 |
| AN16195 | F | 73 | | AD Braak 5 |
| AN02773 | F | 81 | | AD Braak 5 |
| AN00704 | F | 82 | 77.6 ± 6.58 | Control |
| AN00316 | F | 75 | | Control |
| AN17896 | M | 69 | | Control |
| AN12667 | M | 86 | | Control |
| AN08396 | M | 76 | | Control |

f. Cell Lysis and Nuclear Fractionation

A PBS solution of 5×10⁶ Human SH-SY5Y neuroblastoma cells was centrifuged and the resultant cell pellet was resuspended and washed twice with cold PBS. After the second wash, the cell pellet was resuspended in 500 μl of hypotonic buffer (20 mM Tris-Cl, pH 7.4, 10 mM NaCl, 3 mM MgCl2, and 1 mM PMSF) and incubated on ice for 15 min. Next, 25 μl of 10% NP-40 was added to the cell suspension and the sample was vortexed for 10 s. The homogenate was centrifuged for 10 min at 1000×g at 4° C. to obtain the cytoplasmic fraction (supernatant) and nuclear fraction (pellet). After removing the supernetant, the nuclear pellet was resuspended in 50 μl of complete cell extraction buffer (100 mM Tris-Cl, pH 7.4, 2 mM Na₃VO₄, 100 mM NaCl, 1% Triton X-100, 1 mM EDTA, 10% glycerol, 1 mM EGTA, 0.1% SDS, 1 mM NaF, 0.5% deoxycholate, 20 mM Na₄P₂O₇, and 1 mM PMSF) and incubated on ice for 30 min with vortexing at 10 min intervals. The nuclear lysate was centrifuged at 14,000×g for 30 min at 4° C. to obtain the nuclear fraction (supernatant). Protein concentration was measured using the Coomassie Plus (Bradford) Protein Assay kit (Pierce).

g. Immunoblot Analysis

Protein in samples of the supernetant and nuclear fractions was separated by SDSPAGE in a 4-20% gradient Tris-Glycine gel (Invitrogen). The protein was then transferred to nitrocellulose membrane. The membrane was blocked with BSA at room temperature for 15 min and incubated with primary antibody overnight at 4° C. After incubation, the membrane was washed thrice (3×) with TBS-T (Tris-buffered saline-Tween 20) and further incubated with alkaline-phosphatase-conjugated secondary antibody (Jackson Immunoresearch Laboratories) at 1:10,000 dilution for 45 min. Following incubation, the membrane was washed 3× with TBS-T and developed using the 1-step NBT-BCIP substrate (Pierce). Lamin B was used as the nuclear loading control and β-actin as the cytosolic loading control. The immunoblot proteins were detected using ImageQuant RT-ECL (GE Life Sciences) and densitometric quantification was performed using IMAL software, which was developed at our institution. For translocation assays, HDAC translocation to the nucleus was represented as the percentage of total protein in the nucleus [nucleus/(cytosol−nucleus)].

h. Immunofluorescence and Confocal Microscopy

Human SH-SY5Y neuroblastoma cells were grown in eight-chambered slides (Nunc). For immunofluorescence staining, the cells were washed with PBS, pH 7.4, and fixed with 4% paraformaldehyde for 4 min. After fixation, cells were blocked and permeabilized with 5% serum and 0.3% Triton X-100 in 1× PBS for 30 min. Cells were washed three times with 1× PBS and incubated with primary antibodies for 3 h at 1:100 dilution. After incubation, slides were again washed with 1× PBS, three times, and then incubated with FITC anti-rabbit IgG for 1 h at 1:400 dilution. After incubation, the cells were washed and mounted using Pro Long Gold antifade mounting solution (Invitrogen). Stained cells were viewed under an LSM 710 Meta confocal microscope (Zeiss) using excitation wavelengths of 350 and 488 nm and measuring the emission for DAPI (a DNA stain) and FITC at 470 and 525 nm, respectively. Approximately five to six individual cells from each of the eight independent wells were analyzed at magnification of 63×, using Zen 2009 (Zeiss). To measure the percentage of total protein in the nucleus, the whole neuron cell body and nucleus were separately selected as regions of interest. Mean fluorescence intensity in each channel, DAPI, and HDAC, were measured for the nucleus and the whole neuron. Percentage HDAC in nucleus is represented as HDAC (normalized against DAPI) in nucleus/HDAC (normalized against DAPI) in the whole cell body.

i. Knock-Down and Overexpression

Silencing of LRP-1 by RNAi was carried out by transfecting the double-stranded siRNA oligonucleotide, Trilencer-27, which was designed and synthesized by Origene into human SH-SY5Y neuroblastoma cells. Control transfections included both a proven non-targeting siRNA's provided by Origene as well as a non-oligonucleotide control containing only the transfection reagent. PKCε knockdown was performed using 33 nM three target-specific 19-25 nucleotide PKCε siRNA constructs from Santa Cruz Biotechnology. Overexpression of PKCε was achieved by transfecting a pCMV6-ENTRY vector containing human PKCε cDNA (Origene). Transfection was performed using Lipofectamine 2000 following instructions provided by the manufacturer (Invitrogen). Medium was changed 6 h after addition of lipofectamine. LRP-1 and PKC expression were measured 72 h after transfection.

j. qRT-PCR

qRT-PCR was performed and the results analyzed as described previously (Schmittgen et al., Analyzing real-time PCR data by the comparative C(T) method, Nat. Protoc., 3:1101-1108, 2008; Sen et al., 2012 supra). Total RNA (500 ng) was reverse transcribed using oligo(dT) and Superscript III (Invitrogen) at 50° C. for 1 h. The cDNA products were analyzed using a LightCycler 480 II (Roche) PCR machine and LightCycler 480 SYBR Green 1 master mix following the manufacturer's protocol. Primers for PKCε (forward primer: TGGCTGACCTTGGTGTTACTCC, reverse primer: GCTGACTTGGATCGGTCGTCTT, PKCα (forward-ACAACCTGGACAGAGTGAAACTC, reverse: CTTGATGGCGTACAGTTCCTCC), PKC (forward: ACATTCTGCGGCACTCCTGACT, reverse: CCGATG AGCATTTCGTACAGGAG), GAPDH (forward: GTCTCCTCTGACTTCAACAGCG, reverse: ACCACCCTGTTGCTGTAGCCAA), and BDNF (forward: CATCCGAGGACAAGGTGGCTTG, reverse: GCCGAACTTTCTGGTCCTCATC; Origene).

BDNF promoter- and exon specific primers were used as described previously (Pruunsild et al., Dissecting the human BDNF locus: bidirectional transcription, complex splicing, and multiple promoters, Genomics, 90:397-406, 2007). BDNF-promotor I (PI) (forward: GGCACGAACTTTTCTAAGAAG, reverse: CCGCTTTAATAATAATACCAG), BDNF-promotor II (PII) (forward: GAGTCCATTCAGCACCTTGGA, reverse: ATCTCAGTGTGAGCCGAACCT), BDNF-promotor III (PIII) (forward: AGAATCAGGCGGTGGAGGTGGTGTG, reverse: AACCCTCTAAGCCAGCGCCCGAAAC), BDNF-promoter (IV) (PIV) (forward: AAGCATGCAATGCCCTGGAAC, reverse: TGCCTTGACGTGCGCTGTCAT), BDNF-promoter IX (PIX) (forward: CACTTGCAGTTGTTGCTTA, reverse: GGCTTCAAGTTCTCCTTCTTCCCA) were from Invitrogen. BDNF exons were amplified using BDNF exon-specific forward primer (BDNF-exon III forward: AGTTTCGGGCGCTGGCTTAGAG; exon IV forward: GCTGCAGAACAGAAGGAGTACA) and exon IX reverse primers (exon IX reverse: GTCCTCATCCAACAGCTCTTCTATC).

k. ChIP

ChIP was conducted using the SimpleChlP Enzymatic Chromatin IP kit (Cell Signaling Technology) following the manufacturer's protocol. Immunoprecipitations were performed at 4° C. overnight with primary antibodies (HDAC4, HDAC6, or IgG antibody as a control). Immunoprecipitated DNA was subjected to real-time qRT-PCR using primers specific to the human BDNF promoters. The cumulative fluorescence for each amplicon was normalized to input DNA. Products of ChIP-PCR were separated on a 2% agarose gel with ethidium bromide (Invitrogen) to verify amplification.

l. Statistical Analysis

All experiments were performed at least in triplicate, as noted in the figure legends. For confocal images, six or more random fields from three independent experiments were considered for analysis. Data are presented as mean±SEM. All data were analyzed by one-way ANOVA and Newman-Keuls multiple-comparison post-test. Significantly different groups were further analyzed by Student's t test using GraphPad Prism 6 software. p-values<0.05 were considered statistically significant.

What is claimed is:

1. A method for treating a neurodegenerative disorder in a subject comprising:
obtaining a biological sample from the subject;
identifying whether the subject is a carrier of the ApoE4 allele; and
administering to the subject, if the subject is a carrier of the ApoE4 allele, a therapeutically effective amount of a PKC activator.

2. The method of claim 1, wherein the neurodegenerative disorder is chosen from Alzheimer's disease, chronic traumatic encephalopathy (CTE), Parkinson's disease, multiple sclerosis, and traumatic brain injury.

3. The method of claim 1, wherein the neurodegenerative disorder is Alzheimer's disease.

4. The method of claim 3, wherein the Alzheimer's disease is sporadic Alzheimer's disease or late-onset Alzheimer's disease.

5. The method of claim 1, wherein the biological sample is chosen from skin cells, fibroblasts, blood cells, olfactory neurons, and buccal mucosal cells.

6. The method of claim 1, wherein the PKC activator is chosen from macrocyclic lactones, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

7. The method of claim 6, wherein the macrocyclic lactone is bryostatin.

8. The method of claim 7, wherein the bryostatin is chosen from bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18.

9. The method of claim 1, wherein the PKC activator is administered to the subject at a dose of about 5-20 μg/sq·m/week.

10. The method of claim 9, wherein the PKC activator is administered every week for a period of time ranging from about two weeks to about 4 weeks.

11. The method of claim 1, wherein the subject is a homozygous carrier of the Apolipoprotein E ε4 allele.

12. The method of claim 1, wherein the subject is a heterozygous carrier of the Apolipoprotein E ε4 allele.

13. A method for assessing treatment efficacy of a neurodegenerative disease in a subject comprising:
administering to the subject with a neurodegenerative disease one or more therapeutically effective active agent;
obtaining a first biological sample and a second biological sample from the subject, wherein the first and second biological samples are obtained at different time points during the treatment;
measuring the level of PKC-ε in the first and second samples; and
comparing the levels of PKC-ε in the first and second samples, wherein a higher level of PKC-ε in the second sample compared to the first sample is an indicator of efficacy of the treatment.

14. The method of claim 13, wherein the first biological sample is obtained before administering treatment, and the second biological sample is obtained after administering treatment.

15. The method of claim 14, wherein the administration of treatment is chosen from 2 weeks, 3 weeks, 4 weeks, 5 weeks, and 6 weeks.

16. The method of claim 13, wherein the active agent is a PKC activator.

17. The method of claim 16, wherein the PKC activator is chosen from macrocyclic lactones, bryologs, diacylglcerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

18. The method of claim 17, wherein macrocyclic lactone is bryostatin.

19. A method for diagnosing a neurodegenerative disorder in a subject comprising:
obtaining a biological sample form the subject;
lysing the biological sample to obtain a lysate;
differentially fractionating the lysate to obtain a cytoplasmic fraction and a nuclear fraction;
measuring the ratio of HDAC4 or HDAC6 to total HDAC in the nuclear fraction, wherein the subject has neurodegenerative disorder if the ratio of HDAC4 to total nuclear HDAC or the ratio of HDAC6 to total nuclear HDAC is in the range from 0.5 to 0.95.

20. The method of claim 19, wherein the neurodegenerative disorder is chosen from Alzheimer's disease, chronic traumatic encephalopathy (CTE), Parkinson's disease, multiple sclerosis, and traumatic brain injury.

21. A method for assessing a risk of developing a neurodegenerative condition in a subject comprising:
obtaining a biological sample from the subject;
lysing the biological sample to obtain a lysate;
differentially fractionating the lysate to obtain a cytoplasmic fraction and a nuclear fraction;
measuring the level of a HDAC4 or a HDAC6 in the cytoplasmic fraction and the nuclear fraction;
wherein a greater level of HDAC4 or HDAC6 in the nuclear fraction than the cytoplasmic fraction indicates a higher risk of developing the neurodegenerative condition.

22. The method of claim 20, wherein the level of HDAC4 or HDAC6 in the nuclear fraction is 1.5-fold to 2.5 fold greater than the level of HDAC4 or HDAC6 in the cytoplasmic fraction.


SEQUENCE LISTING (23 sequences)

SEQ ID NO: 1 - DNA, 22 bp, Artificial Sequence
  Description of Artificial Sequence Synthetic primer
tggctgaccttggtgttactcc

SEQ ID NO: 2 - DNA, 22 bp, Artificial Sequence
  Description of Artificial Sequence Synthetic primer
gctgacttggatcggtcgtctt

SEQ ID NO: 3 - DNA, 23 bp, Artificial Sequence
  Description of Artificial Sequence Synthetic primer
acaacctggacagagtgaaactc

SEQ ID NO: 4 - DNA, 22 bp, Artificial Sequence
  Description of Artificial Sequence Synthetic primer
cttgatggcgtacagttcctcc

SEQ ID NO: 5 - DNA, 22 bp, Artificial Sequence
  Description of Artificial Sequence Synthetic primer
acattctgcggcactcctgact

SEQ ID NO: 6 - DNA, 23 bp, Artificial Sequence
  Description of Artificial Sequence Synthetic primer
ccgatgagcatttcgtacaggag

SEQ ID NO: 7 - DNA, 22 bp, Artificial Sequence
  Description of Artificial Sequence Synthetic primer
gtctcctctgacttcaacagcg

SEQ ID NO: 8 - DNA, 22 bp, Artificial Sequence
  Description of Artificial Sequence Synthetic primer
accaccctgttgctgtagccaa

SEQ ID NO: 9 - DNA, 22 bp, Artificial Sequence
  Description of Artificial Sequence Synthetic primer
catccgaggacaaggtggcttg

SEQ ID NO: 10 - DNA, 22 bp, Artificial Sequence
  Description of Artificial Sequence Synthetic primer
gccgaactttctggtcctcatc

SEQ ID NO: 11 - DNA, 21 bp, Artificial Sequence
  Description of Artificial Sequence Synthetic oligonucleotide
ggcacgaacttttctaagaag

SEQ ID NO: 12 - DNA, 21 bp, Artificial Sequence
  Description of Artificial Sequence Synthetic oligonucleotide
ccgctttaataataataccag

SEQ ID NO: 13 - DNA, 21 bp, Artificial Sequence
  Description of Artificial Sequence Synthetic oligonucleotide
gagtccattcagcaccttgga

SEQ ID NO: 14 - DNA, 21 bp, Artificial Sequence
  Description of Artificial Sequence Synthetic oligonucleotide
atctcagtgtgagccgaacct

SEQ ID NO: 15 - DNA, 25 bp, Artificial Sequence
  Description of Artificial Sequence Synthetic oligonucleotide
agaatcaggcggtggaggtggtgtg

SEQ ID NO: 16 - DNA, 25 bp, Artificial Sequence
  Description of Artificial Sequence Synthetic oligonucleotide
aaccctctaagccagcgcccgaaac

SEQ ID NO: 17 - DNA, 21 bp, Artificial Sequence
  Description of Artificial Sequence Synthetic oligonucleotide
aagcatgcaatgccctggaac

SEQ ID NO: 18 - DNA, 21 bp, Artificial Sequence
  Description of Artificial Sequence Synthetic oligonucleotide
tgccttgacgtgcgctgtcat

SEQ ID NO: 19 - DNA, 19 bp, Artificial Sequence
  Description of Artificial Sequence Synthetic oligonucleotide
cacttgcagttgttgctta

SEQ ID NO: 20 - DNA, 24 bp, Artificial Sequence
  Description of Artificial Sequence Synthetic oligonucleotide
ggcttcaagttctccttcttccca

SEQ ID NO: 21 - DNA, 22 bp, Artificial Sequence
  Description of Artificial Sequence Synthetic primer
agtttcgggcgctggcttagag

SEQ ID NO: 22 - DNA, 22 bp, Artificial Sequence
  Description of Artificial Sequence Synthetic primer
gctgcagaacagaaggagtaca

SEQ ID NO: 23 - DNA, 25 bp, Artificial Sequence
  Description of Artificial Sequence Synthetic primer
gtcctcatccaacagctcttctatc